(19) United States
(12) Patent Application Publication
(10) Pub. No.: US 2024/0245647 A1
(43) Pub. Date: Jul. 25, 2024

(54) ISOMEROHYDROLASE INHIBITOR FOR TREATMENT OF ATROPHIC FORM OF AGE-RELATED MACULAR DEGENERATION AND STARGARDT DISEASE

(71) Applicants: The Trustees of Columbia University in the City of New York, New York, NY (US); Albany College of Pharmacy and Health Sciences in the City of Albany, Albany, NY (US); The Board of Regents of the University of Oklahoma, Norman, OK (US)

(72) Inventors: Konstantin Petrukhin, New Windsor, NY (US); Christopher L. Cioffi, Albany, NY (US); Yuhong Wang, Edmond, OK (US); Jian-xing Ma, Edmond, OK (US); Gennadiy Moiseyev, Edmond, OK (US)

(73) Assignees: The Trustees of Columbia University in the City of New York, New York, NY (US); Albany College of Pharmacy and Health Sciences in the City of Albany, Albany, NY (US); The Board of Regents of the University of Oklahoma, Norman, OK (US)

(21) Appl. No.: 18/543,655

(22) Filed: Dec. 18, 2023

Related U.S. Application Data
(63) Continuation of application No. PCT/US2022/033891, filed on Jun. 16, 2022.
(60) Provisional application No. 63/212,384, filed on Jun. 18, 2021.

(51) Int. Cl. A61K 31/404; A61K 31/145; A61K 31/343; A61K 31/351; A61K 31/423; A61P 27/02; C07C 317/32; C07D 209/08; C07D 263/56; C07D 305/06; C07D 307/79; C07D 309/06; C07D 333/54; C07D 405/04; C07D 405/06; C07D 495/04; C12N 9/99

(52) U.S. Cl. CPC A61K 31/404; A61K 31/145; A61K 31/343; A61K 31/351; A61K 31/423; A61P 27/02; C07C 317/32; C07D 209/08; C07D 263/56; C07D 305/06; C07D 307/79; C07D 309/06; C07D 333/54; C07D 405/04; C07D 405/06; C07D 495/04; C12N 9/99

(57) ABSTRACT

The present disclosure provides a compound having the structure:

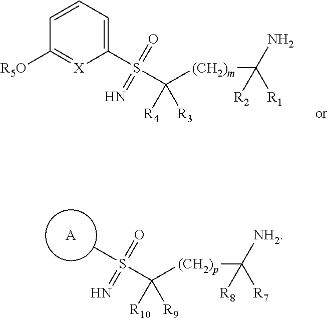

### CROSS-REFERENCE TO RELATED APPLICATIONS

This application is a continuation of PCT International Application No. PCT/US2022/033891, filed Jun. 16, 2022, claiming the benefit of U.S. Provisional Application No. 63/212,384, filed Jun. 18, 2021, the entire contents of each of which are hereby incorporated by reference into the subject application.

Throughout this application, certain publications are referenced in parentheses. Full citations for these publications may be found immediately preceding the claims. The disclosures of these publications in their entireties are hereby incorporated by reference into this application in order to describe more fully the state of the art to which the present disclosure relates.

### BACKGROUND OF THE INVENTION

Age-related macular degeneration (AMD) is the most common cause of blindness in developed countries, particularly in people older than 60 years (Cruickshanks, K. J. et al. 1997; Klein, R. et al. 1999). In the Caucasian population over the age of 80 the frequency of all forms of AMD is 90% in males and 16.4% in females (Cavallotti CAPaC, L. 2008). Age-dependent accumulation of lipofuscin in the retinal pigment epithelium (RPE) is associated with age-related increases of incidences of dry AMD and may be one of several pathogenic factors contributing to the disease onset and progression. Stargardt disease (STGD) is the most common form of inherited macular dystrophy with an estimated prevalence of 1:10,000 (Walia, S. & Fishman, G. A. 2009; Travis, G. H. et al. 2007; Allikmets, R. et al. 1997) and a carrier frequency for mutant ABCA4 alleles reaching 5% in the general population (Jaakson, K. et al. 2003; Roberts, L. J. et al. 2012). The primary biochemical defect in STGD patients is excessive formation of cytotoxic lipofuscin bisretinoids in the retinal pigment epithelium (De Laey, J. J. & Verougstraete, C. 1995; Birnbach, C. D. et al. 1994) due to recessive mutations in the ABCA4 gene. There is no proven treatment for Stargardt disease and dry AMD.

Lipofuscin bisretinoids exert a variety of direct toxic effects on normal RPE cellular processes (Bergmann, M. et al. 2004; Sparrow, J. R. et al. 2003; Dorey, C. K. et al. 1989; Sparrow, J. R. & Boulton, M. 2005). In addition, bisretinoids dysregulate complement system in the retina (Radu, R. A. et al. 2011; Zhou, J. et al. 2006; Zhou, J. et al. 2009; Brandstetter, C. et al. 2015) and induce inflammasome activation in the RPE. Given that excessive production of lipofuscin bisretinoids represents the sole biochemical trigger of STGD and a significant contributor to dry AMD pathogenesis, pharmacological inhibition of bisretinoid synthesis is a disease-modifying approach to STGD and dry AMD treatment (Kennedy, C. J. et al. 1995; Radu, R. A. et al. 2005; Radu, R. A. et al. 2003; Maeda, A. et al. 2006; Palczewski, K. 2010).

Cytotoxic lipofuscin bisretinoids are formed in the retina in a non-enzymatic way from visual cycle retinaldehydes (Sparrow, J. R. et al. 2003; Boyer, N. P. et al. 2012). Given that bisretinoids are synthesized over the course of the properly functioning visual retinoid cycle (Sparrow, J. R. et al. 2003), partial visual cycle inhibition was suggested as a treatment strategy for dry AMD and STGD (Radu, R. A. et al. 2003; Maiti, P. et al. 2006; Maeda, A. et al. 2008). A critical step in the visual cycle is the conversion of all-trans-retinyl ester to 11-cis-retinol in the isomerohydrolase (IMH) reaction catalyzed by RPE65 (Radu, R. A. et al. 2003) (FIG. 1A). RPE65 is a well validated drug target for the inhibition of the pathologic increase in lipofuscin bisretinoid synthesis in STGD1 for a number of reasons; 1) the IMH reaction is rate-limiting in the visual cycle function (Jin, M. et al. 2005) thus making RPE65 an optimal drug target for a partial visual cycle inhibition (Gollapalli, D. R. & Rando, R. R. 2004), 2) due to the restricted pattern of RPE65 expression, its specific inhibition is unlikely to result in systemic adverse effects, 3) genetic ablation of RPE65 in mice reduced bisretinoid synthesis and lipofuscin accumulation (Katz, M. L. & Redmond, T. M. 2001), 4) sporadic Leu450Met amino acid substitution in RPE65 found in certain mouse strains led to the reduction of the visual cycle rate (Wenzel, A. et al. 2001) and inhibition of bisretinoid accumulation (Kim, S. R. et al. 2004), 5) RPE65 inactivation in patients with compound heterozygous or homozygous RPE65 mutations resulted in no or little lipofuscin autofluorescence detected (Lorenz, B. et al. 2004) indicating a reduction in lipofuscin bisretinoid synthesis, and 6) pharmacological inhibition of RPE65 reduced bisretinoid formation in the Abca4^{-/-} mouse model of enhanced retinal lipofuscinogenesis (Radu, R. A. et al. 2003; Maeda, A. et al. 2008; Bavik, C. et al. 2015).

While being well-validated, RPE65 remains to be a very difficult drug target as it catalyzes a complex and poorly understood enzymatic reaction. The exact catalytic mechanisms of the IMH reaction are not fully elucidated. Assays that assess RPE65 activity are not scalable; on a limited scale RPE65 assays can be run in a very limited number of labs worldwide. No large-scale screens for RPE65 inhibitors have ever been reported.

The present disclosure describes novel small molecule inhibitors of RPE65 for treatment of macular degeneration and Stargardt Disease.

### SUMMARY OF THE INVENTION

The present disclosure provides a compound having the structure:

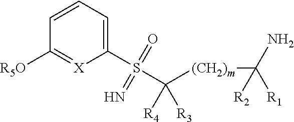

wherein
X is CR₆ or N,
   wherein R₆ is H or halogen;
m represents an integer from 0-2;
R₁ is —H, -(alkyl), -(cycloalkyl) or -D;
R₂ is —H, -(alkyl), -(cycloalkyl) or -D or
R₁ and R₂ together form a —(CH₂)ₙ—, wherein n represents an integer from 2 to 5;
R₃ is —H, -(alkyl), -(cycloalkyl) or -D or
R₁ and R₃ together form a —(CH₂)ₒ—, wherein o represents an integer from 1 to 4;
R₄ is —H, -(alkyl), -(cycloalkyl) or -D; and
R₅ is —H, -(alkyl), -(alkylcycloalkyl) or -(alkylheterocycloalkyl),
   or a pharmaceutically acceptable salt thereof.

The present disclosure also provides a compound having the structure:

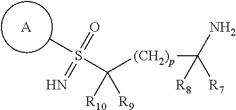

wherein
p represents an integer from 0-2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or D; and
A is

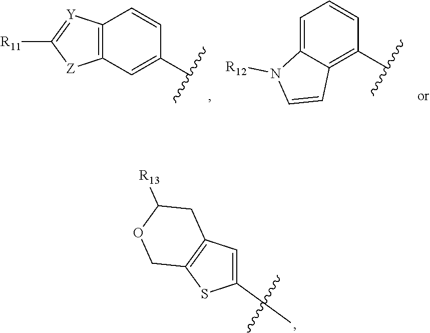

wherein
Y is CH or N,
Z is NR₁₄, O or S,
   wherein R₁₄ is H, -(alkyl), -(cycloalkyl), -(alkylcycloalkyl) or -(alkylheterocycloalkyl);
R₁₁ is H, -(alkyl), -(cycloalkyl), -(alkylcycloalkyl) or -(alkylheterocycloalkyl),
R₁₂ is H, -(alkyl), -(cycloalkyl), -(alkylcycloalkyl) or -(alkylheterocycloalkyl),
R₁₃ is H, -(alkyl), -(cycloalkyl), -(alkylcycloalkyl) or -(alkylheterocycloalkyl),
   or a pharmaceutically acceptable salt thereof.

### BRIEF DESCRIPTION OF THE FIGURES

FIG. 1. Schematic diagram and structure of inhibitor RPE65-61. (FIG. 1A) Schematic diagram representation of the visual cycle and the target of RPE65-61. The visual cycle involves a series of biochemical reactions, one of the key steps of which is the isomerization of all-trans-retinol to 11-cis-retinol by RPE65 isomerase. All-trans-retinol (“ROL”) undergoes a cascade of enzymatic reactions in the retinal pigmented epithelium (RPE), producing a light sensitive chromophore 11-cis-retinal (“RAL”), which binds with opsin to allow photon capture in photoreceptor outer segment. All-trans-RAL has disease implication when accumulated in excessive amounts. RPE65-61 selectively inhibited isomerase activity of RPE65 by competition with all trans-retinyl esters and improves the retinal damage by slowing down the accumulation of all-trans-RAL in LIRD model. (FIG. 1B) RPE65-61 is RPE65 inhibitor, which selectively inhibited isomerase activity of RPE65 by competition with RPE65 substrate all trans-retinyl esters.

FIG. 2. Inhibition of the RPE65 isomerase activity by RPE65-61. Bovine RPE microsomes (20 μg) were incubated with 0.2 μM of all-trans-[3H]-retinol in the presence or absence of RPE65-61 for 1 h at 37° C. The generated retinoids were analyzed by HPLC coupled with flow scintillation analyzer. (FIG. 2A) HPLC elution profile without inhibitor; (FIG. 2B) with 375 nM of RPE65-61. Peak 1, retinyl esters; Peak 2, 11-cis-retinol; Peak 3 all-trans-retinol. (FIG. 2C) RPE65-61 concentration-dependent inhibition of 11-cis-[3H]-retinol generated in the isomerase assay (mean±SEM, n=3).

FIG. 3. Uncompetitive inhibition of RPE65 isomerase by RPE65-61. All-trans-retinyl ester incorporated in liposomes was used as a substrate for purified RPE65 in the isomerase assay. (FIG. 3A) HPLC elution profile of the reaction products without inhibitor; (FIG. 3B) with 180 nM of RPE65-61; Peak 1, retinyl esters; Peak 2, 11-cis-retinol; Peak 3, all-trans-retinol. (FIG. 3C) Lineweaver-Burk plot of 11-cis-retinol generated by RPE65. Liposomes with increasing concentrations (S) of all-trans-retinyl palmitate were incubated with 25 μg of purified chicken RPE65 in the absence (♦) or in the presence of RPE65-61 (180 nM) (▪).

FIG. 4. Systemic injection of inhibitor RPE65-61 delays chromophore regeneration in BALB/cJ mice. Dark-adapted mice were injected with varying amounts of RPE65-61, and subjected to light-adaptation under 5000 lx of fluorescent light for 30 min. The eyes were harvested for HPLC retinoid profiling after 30 min of dark-adaptation, to measure levels of (FIG. 4A) 11-cis-RAL, (FIG. 4B) all-trans-RAL, and (FIG. 4C) all-transretinyl ester. (FIG. 4D) schematic representation of experimental design for RPE65-61 in vivo. Student's t-test. *P<0.05, **<0.01 and ***<0.001. (Mean±SEM).

FIG. 5. LIRD caused the degeneration in the retina and RPE, which was rescued by inhibitor at the dose of 2 mg/kg in BALB/c mice. (FIG. 5A) Representative histological analysis for the retinas of RPE65-61 injected mice 5 days post-LIRD. Retinal cross-sections of No LIRD Control, LIRD+Vehicle, and LIRD+inhibitor RPE65-61 from three groups were stained with H&E for morphological comparison, 40× magnification. (FIG. 5B) Representative OCT images of mice treated with RPE65-61. (FIGS. 5C-5F) Quantification of the protective effects of RPE65-61 from OCT measurements are shown by measuring the average thickness of retina, RPE, outer retina and inner retina. RPE: retinal pigment epithelium; IS: inner segment; OS: outer segment; ONL: outer nuclear layer; OPL: outer plexiform layer; INL: inner nuclear layer; IPL: inner plexiform layer; RNFL: retinal nerve fiber layer. Bars represent mean±SEM. with n=8 per group. Student's t-test. *P<0.05, **<0.01 and ***<0.001, ****<0.0001.

FIG. 6. LIRD caused the degeneration in the retinal photoreceptor cells, which was preserved by inhibitor at the dose of 2 mg/kg in BALB/c mice. (FIGS. 6A-B) Shown are representative images of the Western blot detection of rhodopsin protein expression and the correlating quantifications. (FIGS. 6C-D) Cone density was evaluated by immunofluorescence, representative confocal images of immunofluorescence labeling with Peanut Agglutinin Lectin (PNA) and the correlating quantifications, 40× magnification. Data are represented as mean±SEM. with n=3-8 per group. Student's t-test. *P<0.05, **<0.01 and ***<0.001, ****<0.0001.

FIG. 7. ERG responses of RPE65-61 injected mice 5 days post-LIRD. (FIG. 7A) Representative scotopic A amplitudes from three mouse groups. Seven different stimulus intensities were used, ranging from 0.002 to 400 cd s/m². (FIG. 7B) Comparison of the averaged scotopic B-wave amplitude from the three groups. (FIG. 7C) Representative photopic B amplitude from the three groups: No LIRD control, LIRD+Vehicle, or LIRD+RPE65-61, Student's t-test was used (n=8, mean±SEM, *P<0.05, **<0.01 and ***<0.001).

FIG. 8. Elevated protein levels of cGAS-STING pathway in BALB/c mice. The CGAS, STING and phosphorylated NF-κB (pp65). (FIGS. 8A-8C) Retinas were harvested and cGAS and STING protein levels were measured by Western blot analysis with β-actin as loading control and densitometry quantification. (FIGS. 8D-8E) Levels of phosphorylated NF-κB (p-NF-κB) were determined by Western blotting. Total NF-κB (p65) was used as an internal control and densitometry quantification. Each lane represents an individual mouse. (FIGS. 8F-8G) Photoreceptor cell apoptosis was evaluated by TUNEL on the retinal sections at the dose of 2 mg/kg in BALB/c mice and correlating quantitative analysis. Student's t-test was used (n=5, mean±SEM, *P<0.05, **<0.01).

FIG. 9. The structure of RPE65-61 and (R)-emixustat with the IC₅₀ values in the IMH assay shown.

FIG. 10. (FIG. 10A) Structure of RPE65-76; (FIG. 10B) Compound stability in dog plasma (reflecting the propensity to VAP-1-mediated metabolism).

FIG. 11. Sequestration of all-trans-retinal (“atRAL”) in vitro by RPE65-61 and its analogues. Test compounds (100 μM) were incubated with equimolar amounts of all-trans-retinal for 30 min. The UV-vis spectra were collected and the concentration of all-trans-retinal was assessed based on the 370 nm absorbance. (FIG. 11A) Graph bars show mean atRAL levels normalized to DMSO; error bars show SD; one-way ANOVA with Holm-Sidak post-hoc test was used for statistical analysis; *P<0.05; ****P<0.0001. (FIG. 11B) Compound structures with potency in the primary RPE65 inhibition assay shown.

### DETAILED DESCRIPTION OF THE INVENTION

The present disclosure provides a compound having the structure:

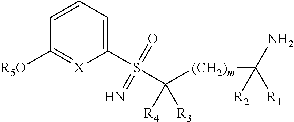

wherein
X is CR₆ or N,
   wherein R₆ is H or halogen;
m represents an integer from 0-2;
R₁ is —H, -(alkyl), -(cycloalkyl) or -D;
R₂ is —H, -(alkyl), -(cycloalkyl) or -D or
R₁ and R₂ together form a —(CH₂)ₙ—, wherein n represents an integer from 2 to 5;
R₃ is —H, -(alkyl), -(cycloalkyl) or -D or
R₁ and R₃ together form a —(CH₂)ₒ—, wherein o represents an integer from 1 to 4;
R₄ is —H, -(alkyl), -(cycloalkyl) or -D; and
R₅ is —H, -(alkyl), -(alkylcycloalkyl) or -(alkylheterocycloalkyl),
   or a pharmaceutically acceptable salt thereof.

In an embodiment, the compound having the structure:

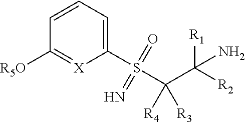

wherein
X is CR₆ or N,
   wherein R₆ is H or halogen;
R₁ is —H, -(alkyl), -(cycloalkyl) or -D;
R₂ is —H, -(alkyl), -(cycloalkyl) or -D or
R₁ and R₂ together form a —(CH₂)ₙ—, wherein n represents an integer from 2 to 5;
R₃ is —H, -(alkyl), -(cycloalkyl) or -D or
R₁ and R₃ together form a —(CH₂)ₒ—, wherein o represents an integer from 1 to 4;
R₄ is —H, -(alkyl), -(cycloalkyl) or -D; and
R₅ is —H, -(alkyl), -(alkylcycloalkyl) or -(alkylheterocycloalkyl),
   or a pharmaceutically acceptable salt thereof.

In some embodiments, the compound wherein
X is CR₆ or N,
   wherein R₆ is H or halogen;
R₁ is —H, -(alkyl), -(cycloalkyl) or -D;
R₂ is —H, -(alkyl), -(cycloalkyl) or -D;
R₃ is —H, -(alkyl), -(cycloalkyl) or -D;
R₄ is —H, -(alkyl), -(cycloalkyl) or -D; and
R₅ is —H, -(alkyl), -(alkylcycloalkyl) or -(alkylheterocycloalkyl),
   or a pharmaceutically acceptable salt thereof.

In some embodiments, the compound wherein
X is CR₆ or N,
   wherein R₆ is H or F;
m represents an integer from 0-2;
R₁ is —H, —CH₃ or -D;
R₂ is —H, —CH₃ or -D or
R₁ and R₂ together form a —(CH₂)ₙ—, wherein n represents an integer from 2 to 5;
R₃ is —H, —CH₃ or -D or
R₁ and R₃ together form a —(CH₂)ₒ—, wherein o represents an integer from 1 to 4;
R₄ is —H, —CH₃ or -D; and
R₅ is —H,

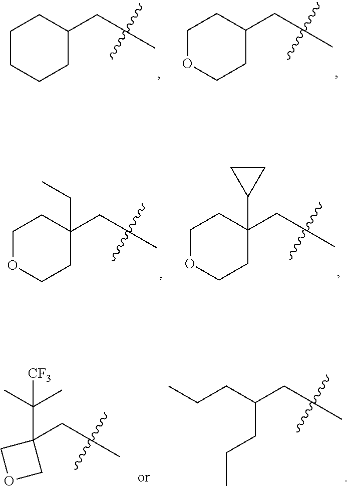

or a pharmaceutically acceptable salt thereof.

In some embodiments, the compound wherein R₁ is H, —CH₃ or D.

In some embodiments, the compound wherein R₂ is H, —CH₃ or D.

In some embodiments, the compound wherein R₃ is H, —CH₃ or D.

In some embodiments, the compound wherein R₄ is H, —CH₃ or D.

In some embodiments, the compound wherein R₁, R₂, R₃ and R₄ are each H.

In some embodiments, the compound wherein R₁, R₂, R₃ and R₄ are each D.

In some embodiments, the compound wherein R₁ is —CH₃, and R₂, R₃ and R₄ are each H.

In some embodiments, the compound wherein
X is CH.

In some embodiments, the compound wherein
X is CF.

In some embodiments, the compound wherein
X is N.

In some embodiments, the compound wherein R₅ is

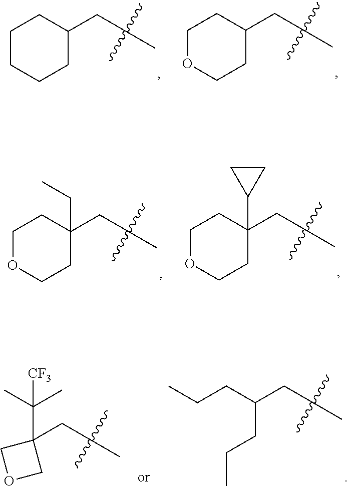

In some embodiments, the compound having the structure:

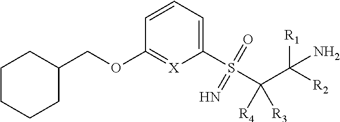

or a pharmaceutically acceptable salt thereof.

In some embodiments, the compound having the structure:

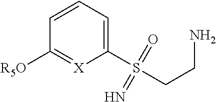

or a pharmaceutically acceptable salt thereof.

In some embodiments, the compound wherein
R₁ and R₂ together form a —(CH₂)ₙ—, wherein n represents an integer from 2 to 5.

In some embodiments, the compound wherein
n is 2.

In some embodiments, the compound having the structure:

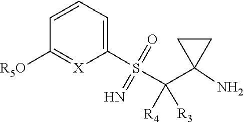

or a pharmaceutically acceptable salt thereof.

In some embodiments, the compound wherein
n is 3.

In some embodiments, the compound having the structure:

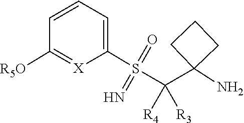

or a pharmaceutically acceptable salt thereof.

In some embodiments, the compound wherein
n is 4.

In some embodiments, the compound having the structure:

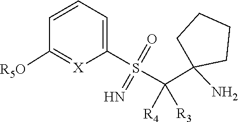

or a pharmaceutically acceptable salt thereof.

In some embodiments, the compound wherein
n is 5.

In some embodiments, the compound having the structure:

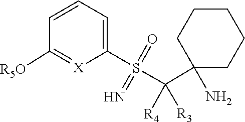

or a pharmaceutically acceptable salt thereof.

In some embodiments, the compound wherein
R₁ and R₃ together form a —(CH₂)ₒ—, wherein o represents an integer from 1 to 4.

In some embodiments, the compound wherein
m is 1 and o is 1.

In some embodiments, the compound having the structure:

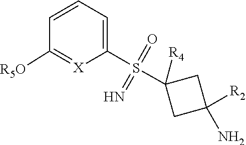

or a pharmaceutically acceptable salt thereof.

In some embodiments, the compound wherein
m is 1 and o is 2.

In some embodiments, the compound having the structure:

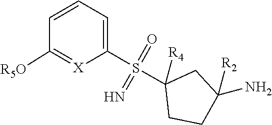

or a pharmaceutically acceptable salt thereof.

In some embodiments, the compound wherein
m is 1 and o is 3.

In some embodiments, the compound having the structure:

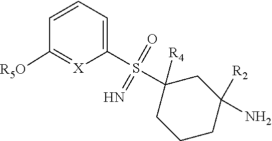

or a pharmaceutically acceptable salt thereof.

In some embodiments, the compound wherein
m is 1 and o is 4.

In some embodiments, the compound having the structure:

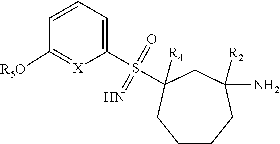

or a pharmaceutically acceptable salt thereof.

In some embodiments, the compound having the structure:

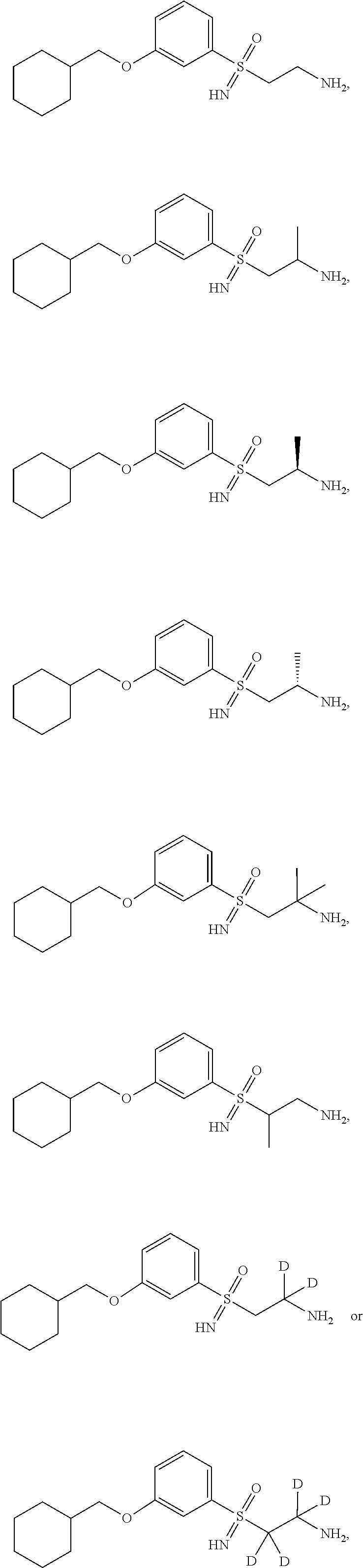

or a pharmaceutically acceptable salt thereof.

In some embodiments, the compound having the structure:

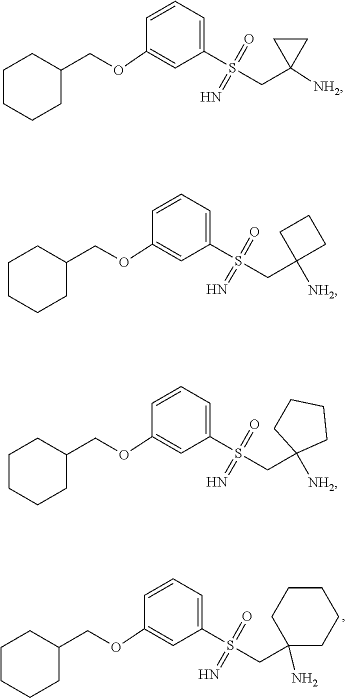 [SMILES: N=S(=O)(CC1(N)CC1)c1cccc(OCC2CCCCC2)c1 ; N=S(=O)(CC1(N)CCC1)c1cccc(OCC2CCCCC2)c1 ; N=S(=O)(CC1(N)CCCC1)c1cccc(OCC2CCCCC2)c1 ; N=S(=O)(CC1(N)CCCCC1)c1cccc(OCC2CCCCC2)c1]

or a pharmaceutically acceptable salt thereof.

In some embodiments, the compound having the structure:

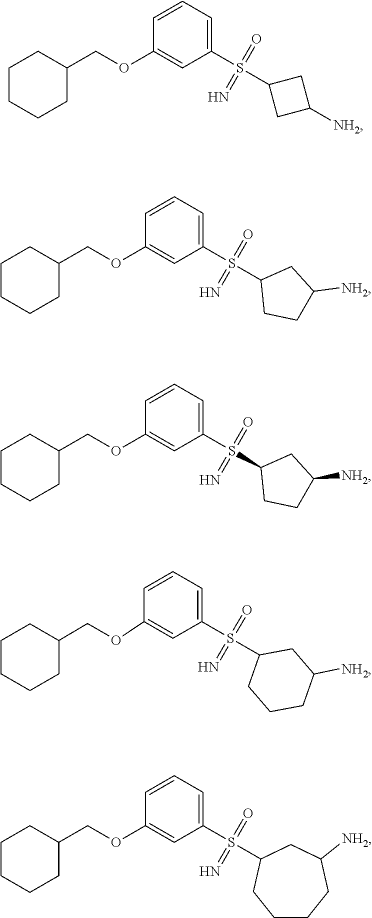 [SMILES: N=S(=O)(c1cccc(OCC2CCCCC2)c1)C1CC(N)C1 ; N=S(=O)(c1cccc(OCC2CCCCC2)c1)C1CCC(N)C1 ; N=S(=O)(c1cccc(OCC2CCCCC2)c1)[C@@H]1CC[C@H](N)C1 ; N=S(=O)(c1cccc(OCC2CCCCC2)c1)C1CCCC(N)C1 ; N=S(=O)(c1cccc(OCC2CCCCC2)c1)C1CCCCC(N)C1]

or a pharmaceutically acceptable salt thereof.

In some embodiments, the compound having the structure:

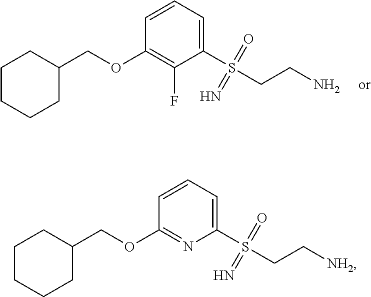 [SMILES: N=S(=O)(CCN)c1cccc(OCC2CCCCC2)c1F ; N=S(=O)(CCN)c1cccc(OCC2CCCCC2)n1]

or a pharmaceutically acceptable salt thereof.

In some embodiments, the compound having the structure:

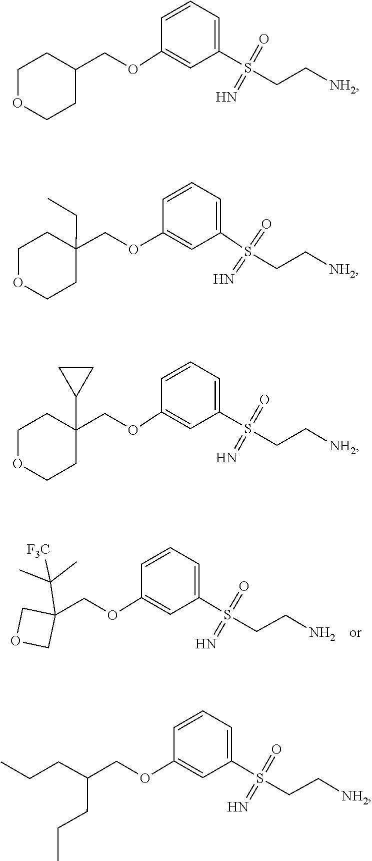 [SMILES: N=S(=O)(CCN)c1cccc(OCC2CCOCC2)c1 ; CCC1(COc2cccc(S(=N)(=O)CCN)c2)CCOCC1 ; N=S(=O)(CCN)c1cccc(OCC2(C3CC3)CCOCC2)c1 ; CC(C)(C(F)(F)F)C1(COc2cccc(S(=N)(=O)CCN)c2)COC1 ; CCCC(CCC)COc1cccc(S(=N)(=O)CCN)c1]

or a pharmaceutically acceptable salt thereof.

The present disclosure provides a compound having the structure:

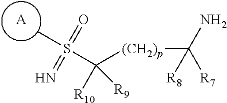

wherein
p represents an integer from 0-2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
A is

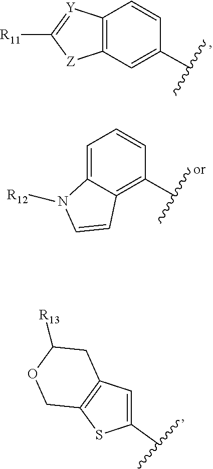

wherein
Y is CH or N,
Z is NR₁₄, O or S,
   wherein R₁₄ is H, -(alkyl), -(cycloalkyl), -(alkylcycloalkyl) or -(alkylheterocycloalkyl);
R₁₁ is H, -(alkyl), -(cycloalkyl), -(alkylcycloalkyl) or -(alkylheterocycloalkyl),
R₁₂ is H, -(alkyl), -(cycloalkyl), -(alkylcycloalkyl) or -(alkylheterocycloalkyl),
R₁₃ is H, -(alkyl), -(cycloalkyl), -(alkylcycloalkyl) or -(alkylheterocycloalkyl),
   or a pharmaceutically acceptable salt thereof.

In an embodiment, the compound having the structure:

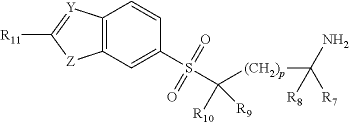

wherein
Y is CH or N;
Z is NR₁₄, O or S,
   wherein R₁₄ is H, -(alkyl), -(cycloalkyl), -(alkylcycloalkyl) or -(alkylheterocycloalkyl);
p represents an integer from 0-2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₁ is H, -(alkyl), -(cycloalkyl), -(alkylcycloalkyl) or -(alkylheterocycloalkyl),
   or a pharmaceutically acceptable salt thereof.

In some embodiments, the compound having the structure:

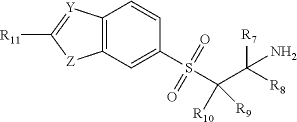

wherein
Y is CH or N;
Z is NR₁₄, O or S,
   wherein R₁₄ is H, -(alkyl), -(alkylcycloalkyl) or -(alkylheterocycloalkyl);
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₁ is H, -(alkyl), -(alkylcycloalkyl) or -(alkylheterocycloalkyl),
   or a pharmaceutically acceptable salt thereof.

In some embodiments, the compound wherein
Y is CH or N;
Z is NR₁₄, O or S,
   wherein R₁₄ is H, -(alkyl), -(alkylcycloalkyl) or -(alkylheterocycloalkyl);
R₇ is —H, -(alkyl), -(cycloalkyl) or -D;
R₈ is —H, -(alkyl), -(cycloalkyl) or -D;
R₉ is —H, -(alkyl), -(cycloalkyl) or -D;
R₁₀ is —H, -(alkyl), -(cycloalkyl) or -D; and
R₁₁ is H, -(alkyl), -(alkylcycloalkyl) or -(alkylheterocycloalkyl),
   or a pharmaceutically acceptable salt thereof.

In some embodiments, the compound wherein
Y is CH and Z is NR₁₄.

In some embodiments, the compound wherein
Y is N and Z is O.

In some embodiments, the compound wherein
Y is CH and Z is O.

In some embodiments, the compound wherein
Y is CH and Z is S.

In some embodiments, the compound having the structure:

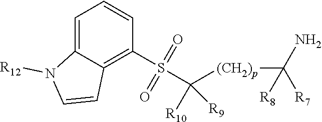

wherein
p represents an integer from 0-2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₂ is H, -(alkyl), -(cycloalkyl), -(alkylcycloalkyl) or -(alkylheterocycloalkyl),
   or a pharmaceutically acceptable salt thereof.

In some embodiments, the compound having the structure:

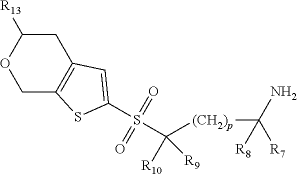

wherein
p represents an integer from 0-2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₃ is H, -(alkyl), -(cycloalkyl), -(alkylcycloalkyl) or -(alkylheterocycloalkyl),
   or a pharmaceutically acceptable salt thereof.

In some embodiments, the compound wherein
p is 0.

In some embodiments, the compound wherein R₇ is H, —CH₃ or D.

In some embodiments, the compound wherein R₈ is H, —CH₃ or D.

In some embodiments, the compound wherein R₉ is H, —CH₃ or D.

In some embodiments, the compound wherein R₁₀ is H, —CH₃ or D.

In some embodiments, the compound wherein R₇, R₈, R₉ and R₁₀ are each H.

In some embodiments, the compound wherein R₇, R₈, R₉ and R₁₀ are each D.

In some embodiments, the compound wherein R₇ is —CH₃, and R₈, R₉ and R₁₀ are each H.

In some embodiments, the compound wherein
R₇ and R₈ together form a —(CH₂)_{q}—, wherein q represents an integer from 2 to 5.

In some embodiments, the compound wherein
q is 2.

In some embodiments, the compound wherein
q is 3.

In some embodiments, the compound wherein
q is 4.

In some embodiments, the compound wherein
q is 5.

In some embodiments, the compound wherein
R₇ and R₉ together form a —(CH₂)ᵣ—, wherein r represents an integer from 1 to 4.

In some embodiments, the compound wherein
p is 1 and r is 1.

In some embodiments, the compound wherein
p is 1 and r is 2.

In some embodiments, the compound wherein
p is 1 and r is 3.

In some embodiments, the compound wherein
p is 1 and r is 4.

In some embodiments, the compound wherein R₁₁ is H, —CH₃,

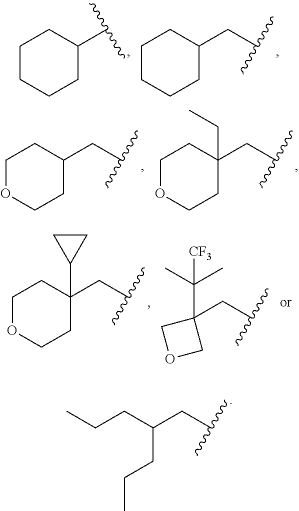

In some embodiments, the compound wherein R₁₂ is H, —CH₃,

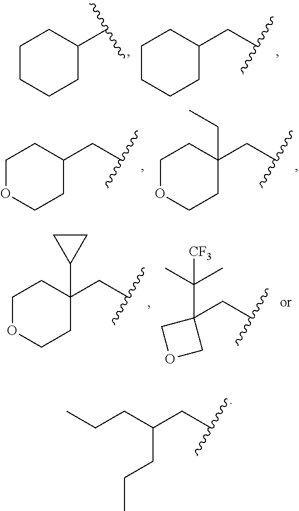

In some embodiments, the compound wherein R₁₃ is H, —CH₃,

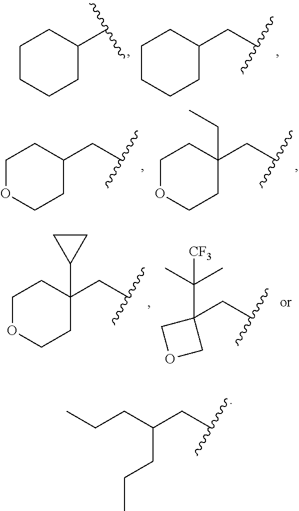

In some embodiments, the compound wherein R₁₄ is H, —CH₃,

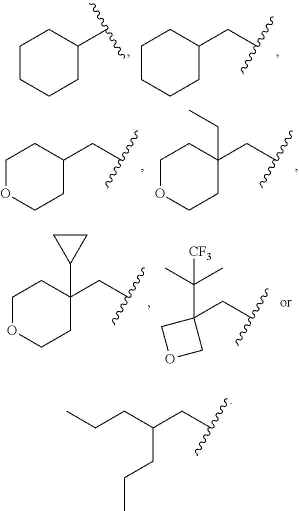

In some embodiments, the compound having the structure:

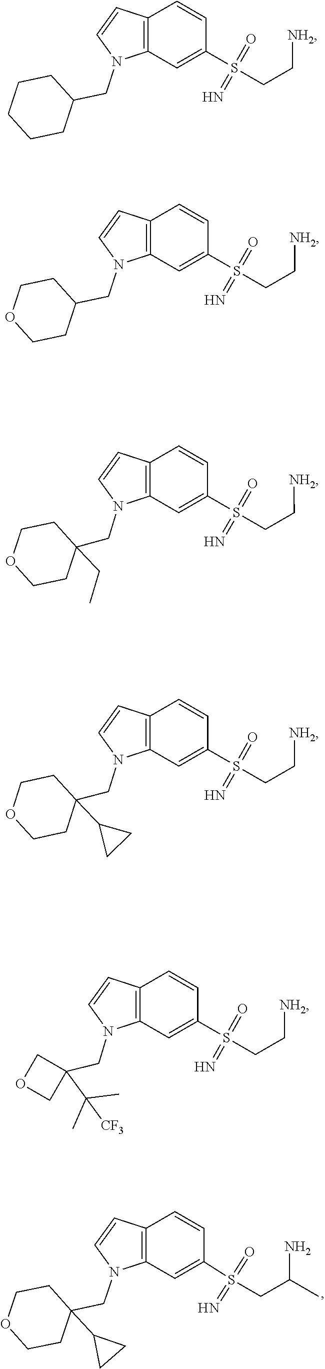 [SMILES: N=S(=O)(CCN)c1ccc2ccn(CC3CCCCC3)c2c1 ; N=S(=O)(CCN)c1ccc2ccn(CC3CCOCC3)c2c1 ; CCC1(Cn2ccc3ccc(S(=N)(=O)CCN)cc32)CCOCC1 ; N=S(=O)(CCN)c1ccc2ccn(CC3(C4CC4)CCOCC3)c2c1 ; CC(C)(C(F)(F)F)C1(Cn2ccc3ccc(S(=N)(=O)CCN)cc32)COC1 ; CC(N)CS(=N)(=O)c1ccc2ccn(CC3(C4CC4)CCOCC3)c2c1]

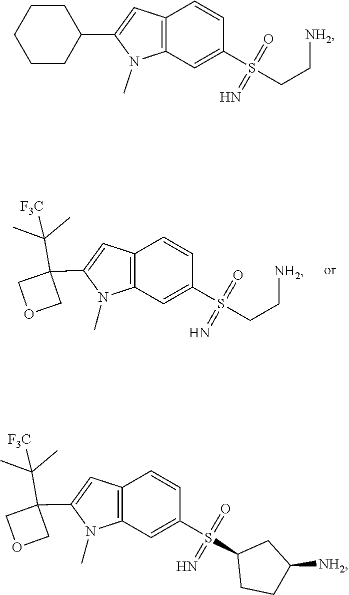 [SMILES: Cn1c(C2CCCCC2)cc2ccc(S(=N)(=O)CCN)cc21 ; Cn1c(C2(C(C)(C)C(F)(F)F)COC2)cc2ccc(S(=N)(=O)CCN)cc21 ; Cn1c(C2(C(C)(C)C(F)(F)F)COC2)cc2ccc(S(=N)(=O)[C@@H]3CC[C@H](N)C3)cc21]

or a pharmaceutically acceptable salt thereof.

In some embodiments, the compound having the structure:

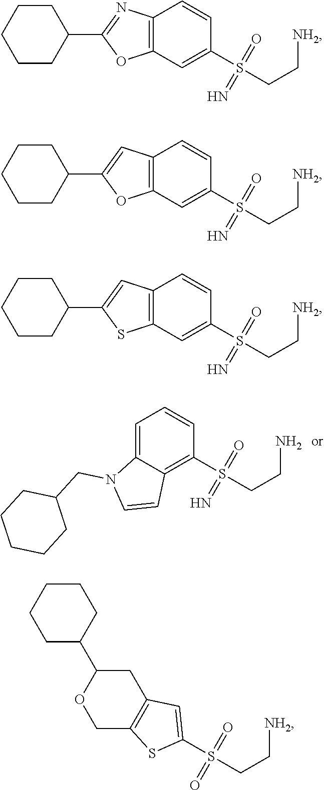

or a pharmaceutically acceptable salt thereof.

The present disclosure also provides a pharmaceutical composition comprising the compound of the present disclosure and a pharmaceutically acceptable carrier.

In some embodiments, a method of inhibiting RPE65 isomerohydrolase comprising contacting the RPE65 isomerohydrolase with a compound of the present disclosure or a composition of the present disclosure.

In some embodiments, a method for treating a disease characterized by excessive lipofuscin accumulation in the retina in a subject afflicted therewith comprising administering to the subject an effective amount of a compound of the present disclosure or a composition of the present disclosure.

In some embodiments, wherein the disease is further characterized by bisretinoid-mediated macular degeneration.

In some embodiments, wherein the amount of the compound of the present disclosure is effective to inhibit RPE65 isomerohydrolase in the subject.

In some embodiments, the method wherein the amount of the compound of the present disclosure is effective to inhibit the conversion of all-trans retinyl ester to 11-cis retinol in the retinal pigment epithelium in the subject.

In some embodiments, the method wherein the amount of the compound of the present disclosure is effective to lower the retinal concentration of a bisretinoid in lipofuscin in the subject.

In some embodiments, the method wherein the bisretinoid is A2E.

In some embodiments, the method wherein the bisretinoid is isoA2E.

In some embodiments, the method wherein the bisretinoid is A2-DHP-PE.

In some embodiments, the method wherein the bisretinoid is atRAL di-PE.

In some embodiments, the method wherein bisretinoid synthesis is reduced through retinaldehyde trapping.

In some embodiments, the method wherein the disease characterized by excessive lipofuscin accumulation in the retina is Age-Related Macular Degeneration.

In some embodiments, the method wherein the disease characterized by excessive lipofuscin accumulation in the retina is dry (atrophic) Age-Related Macular Degeneration.

In some embodiments, the method wherein the disease characterized by excessive lipofuscin accumulation in the retina is Stargardt Disease.

In some embodiments, the method wherein the disease characterized by excessive lipofuscin accumulation in the retina is Best disease.

In some embodiments, the method wherein the disease characterized by excessive lipofuscin accumulation in the retina is adult vitelliform maculopathy.

In some embodiments, the method wherein the disease characterized by excessive lipofuscin accumulation in the retina is Stargardt-like macular dystrophy.

In some embodiments, the method wherein the subject is a mammal.

In some embodiments, the method wherein the compound of the present disclosure or pharmaceutical composition of the present disclosure is administered to the mammal intravenously, intravitreally, or orally.

In some embodiments, the method wherein the compound of the present disclosure or pharmaceutical composition of the present disclosure is administered to the mammal orally.

In some embodiments, the method wherein the compound of the present disclosure or pharmaceutical composition of the present disclosure is administered as an oral dosage in the form of a capsule, or tablet.

In some embodiments, the method wherein the mammal is a human.

In some embodiments of the above methods, the disease characterized by excessive lipofuscin accumulation in the retina is Age-Related Macular Degeneration, is dry (atrophic) Age-Related Macular Degeneration, Stargardt Disease, Best disease, adult vitelliform maculopathy, or Stargardt-like macular dystrophy.

In some embodiments of the above methods, the subject is a mammal. In some embodiments of the above methods, the mammal is a human.

The present disclosure provides a method for treating a disease characterized by excessive lipofuscin accumulation in the retina in a mammal afflicted therewith comprising administering to the mammal an effective amount of a compound of the present disclosure or a composition of the present disclosure.

In some embodiments of the method, wherein the disease is further characterized by bisretinoid-mediated macular degeneration.

In some embodiments of the method, wherein the amount of the compound is effective to lower the serum concentration of RBP4 in the mammal.

In some embodiments of the method, wherein the amount of the compound is effective to lower the retinal concentration of a bisretinoid in lipofuscin in the mammal.

In some embodiments of the method, wherein the bisretinoid is A2E. In some embodiments of the method, wherein the bisretinoid is isoA2E. In some embodiments of the method, wherein the bisretinoid is A2-DHP-PE.

In some embodiments of the method, wherein the bisretinoid is atRAL di-PE.

In some embodiments of the method, wherein bisretinoid synthesis is reduced through retinaldehyde trapping.

In some embodiments of the method, wherein the disease characterized by excessive lipofuscin accumulation in the retina is Age-Related Macular Degeneration.

In some embodiments of the method, wherein the disease characterized by excessive lipofuscin accumulation in the retina is dry (atrophic) Age-Related Macular Degeneration. In some embodiments of the method, wherein the disease characterized by excessive lipofuscin accumulation in the retina is Stargardt Disease. In some embodiments of the method, wherein the disease characterized by excessive lipofuscin accumulation in the retina is Best disease. In some embodiments of the method, wherein the disease characterized by excessive lipofuscin accumulation in the retina is adult vitelliform maculopathy. In some embodiments of the method, wherein the disease characterized by excessive lipofuscin accumulation in the retina is Stargardt-like macular dystrophy. In some embodiments, bisretinoid-mediated macular degeneration is Age-Related Macular Degeneration or Stargardt Disease. In some embodiments, the bisretinoid-mediated macular degeneration is Age-Related Macular Degeneration. In some embodiments, the bisretinoid-mediated macular degeneration is dry (atrophic) Age-Related Macular Degeneration. In some embodiments, the bisretinoid-mediated macular degeneration is Stargardt Disease. In some embodiments, the bisretinoid-mediated macular degeneration is Best disease. In some embodiments, the bisretinoid-mediated macular degeneration is adult vitelliform maculopathy. In some embodiments, the bisretinoid-mediated macular degeneration is Stargardt-like macular dystrophy. The bisretinoid-mediated macular degeneration may comprise the accumulation of lipofuscin deposits in the retinal pigment epithelium.

As used herein, “bisretinoid lipofuscin” is lipofuscin containing a cytotoxic bisretinoid. Cytotoxic bisretinoids include but are not necessarily limited to A2E, isoA2E, atRAL di-PE, and A2-DHP-PE.

Except where otherwise specified, when the structure of a compound of the present disclosure includes an asymmetric carbon atom and/or sulfur atom, it is understood that the compound occurs as a racemate, racemic mixture, and isolated single enantiomer. All such isomeric forms of these compounds are expressly included in the present disclosure. Except where otherwise specified, each stereogenic carbon and sulfur atom may be of the R or S configuration. It is to be understood accordingly that the isomers arising from such asymmetry (e.g., all enantiomers and diastereomers) are included within the scope of the present disclosure, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The present disclosure is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, is intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, is intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds of the present disclosure, the substituents may be substituted or unsubstituted, unless specifically defined otherwise.

In the compounds used in the method of the present disclosure, the substituents may be substituted or unsubstituted, unless specifically defined otherwise.

In the compounds of the present disclosure, alkyl, cycloalkyl, alkylcycloalkyl, alkylheterocycloalkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

In the compounds used in the method of the present disclosure, alkyl, cycloalkyl, alkylcycloalkyl, alkylheterocycloalkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present disclosure can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present disclosure, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “heterocycloalkyl” is intended to mean a 3- to 10-membered nonaromatic ring containing from 1 to 4 heteroatoms selected from the group consisting of O, N and S, and includes bicyclic groups.

The term “alkylcycloalkyl” refers to alkyl groups as described above wherein one or more bonds to hydrogen contained therein are replaced by a bond to a cycloalkyl group as described above. It is understood that an “alkylcycloalkyl” group is connected to a core molecule through a bond from the alkyl group and that the cycloalkyl group acts as a substituent on the alkyl group. An example of an alkylcycloalkyl moiety includes, but is not limited to,

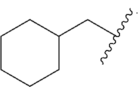 [SMILES: *CC1CCCCC1]

The term “alkylheterocycloalkyl” refers to alkyl groups as described above wherein one or more bonds to hydrogen contained therein are replaced by a bond to a heterocycloalkyl group as described above. It is understood that an “alkylheterocycloalkyl” group is connected to a core molecule through a bond from the alkyl group and that the heterocycloalkyl group acts as a substituent on the alkyl group. Examples of alkylheterocycloalkyl moieties include, but are not limited to,

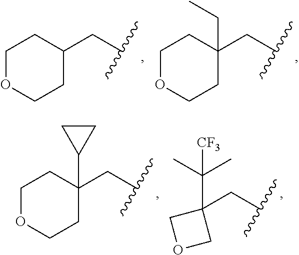

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical, straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙ alkyl” is defined to include groups having 1, 2, . . . , n−1 or n carbons in a linear or branched arrangement (e.g. C₁-C₂ hydroxyalkyl, C₁-C₃ hydroxyalkyl, C₁-C₄ hydroxyalkyl, C₁-C₅ hydroxyalkyl, or C₁-C₆ hydroxyalkyl). For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an —OH group.

As used herein, “heteroalkyl” includes both branched and straight-chain saturated aliphatic hydrocarbon groups having the specified number of carbon atoms and at least 1 heteroatom within the chain or branch.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are: (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, and tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or can contain one or more degrees of unsaturation, and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The term “ester” is intended to mean an organic compound containing the R—O—CO—R′ group.

The term “amide” is intended to mean an organic compound containing the R—CO—NH—R′ or R—CO—N—R′R″ group.

The term “phenyl” is intended to mean an aromatic six membered ring containing six carbons.

The term “benzyl” is intended to mean a —CH₂R₁ group wherein the R₁ is a phenyl group.

The term “tetrahydropyran” is intended to mean a heterocyclyl having a six-membered ring containing five carbon atoms and one O atom.

The term “indoline” is intended to mean a heterobicycle having a five-membered ring fused to a phenyl ring with the five-membered ring containing one nitrogen atom which is directly attached to the phenyl ring. The compound is based on indole, but the 2-3 bond of the 5-membered ring is saturated.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro; nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present disclosure may be prepared by techniques well known in organic synthesis and familiar to a practitioner ordinarily skilled in the art. However, these may not be the only means by which to synthesize or obtain the desired compounds.

The compounds used in the method of the present disclosure may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the present disclosure comprises a compound used in the method of the present disclosure as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present disclosure using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present disclosure may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; and alkali or organic base salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present disclosure. These salts can be prepared in situ during the final isolation and purification of the compounds of the present disclosure, or by separately reacting a purified compound of the present disclosure in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection. Further, an embodiment of “treatment” encompasses “prevention.”

The compounds used in the method of the present disclosure may be administered in various forms, including those detailed herein. The treatment with the compound may be a component of a combination therapy or an adjunct therapy, i.e. the subject or patient in need of the drug is treated with or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as: the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment; and the desired therapeutic effect.

A dosage unit of the compounds used in the method of the present disclosure may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present disclosure can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present disclosure are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present disclosure may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the present disclosure.

The present disclosure will be better understood by reference to the Experimental Details which follow, but those skilled in the art will readily appreciate that the specific experiments detailed are only illustrative of the present disclosure as described more fully in the claims which follow thereafter.

### Experimental Details

### Biology

### Animal Care

All animal studies were approved by the Institutional Animal Care and Use Committee (IACUC) of the University of Oklahoma Health Sciences Center, and performed following the guidelines of the Association for Research in Vision and Ophthalmology (ARVO) statement for the “Use of Animals in Ophthalmic and Vision Research”. BALB/cJ mice were used for all experiments. The animals were maintained in standard housing with 12 h light/dark cycles and food and water ad libitum.

### Example 1. In Vitro Retinol Isomerase Assays

All-trans [11,12-³H]-retinol (1 mCi/ml, 45.5 Ci/mmol, Perkin Elmer, Boston, MA) in N,N-dimethyl formamide (DMF) was used as the substrate for the isomerohydrolase assay. Bovine RPE microsomes were prepared as described previously. For each reaction, 25 μg microsomal proteins from the bovine RPE were added into 200 μl of reaction buffer (10 mM BTP, pH 8.0, 100 mM NaCl) containing 0.2 μM of all-trans retinol, 1% BSA and 25 μM of cellular retinaldehyde-binding protein (CRALBP). For the inhibition of isomerase activity, RPE65-61 dissolved in the DMF was added to the reaction prior to addition of all-trans retinol. The reaction was stopped and retinoids extracted with 300 μl of cold methanol and 300 μl of hexane and centrifuged at 10,000×g for 5 min. The upper layer was collected and the generated retinoids were analyzed by normal phase HPLC as described (Moiseyev, G. et al. 2003). The peak of each retinoid isomer was identified based on its characteristic retention time of retinoid standards. The isomerohydrolase activity was calculated from the area of the 11-cis retinol peak using Radiomatic 610TR software (Perkin Elmer, Boston, MA) with synthetic 11-cis [³H]-retinol as a standard. Alternatively, for each reaction, an equal amount of all-trans retinyl palmitate (atRP) incorporated into the liposomes (250 μM lipids, 3.3 μM atRP) and 25 μg of purified RPE65 were incubated in 200 μl of reaction buffer (10 mM BTP, pH 8.0, 100 mM NaCl) containing 0.5% BSA and 25 μM cellular retinaldehyde-binding protein for 2 hr at 37° C. The retinoid profile after completion of the reaction was analyzed by HPLC where the peaks were identified by co-elution with retinoid standards. The RPE65 isomehydrolase activity was calculated based on the 11-cis retinol peak area as described (Moiseyev, G. et al. 2005; Nikolaeva, O. et al. 2009). Nonlinear regression analysis of v-versus-[S] data was used to calculate Vₘₐₓ (apparent) and Kₘ (apparent) in the absence and in the presence of the inhibitor. The inhibition constant for RPE65-61 was calculated from the following equation: Ki=[I]/(Vₘᵢ/Vₘ−1) where [I] is concentration of inhibitor, Vₘ is maximal velocity in the absence of the inhibitor, and Vₘᵢ is maximal velocity in the presence of the inhibitor.

### Example 2. Light-Induced Retinal Damage (LIRD)

Mice were dark-adapted overnight with food and water ad libitum. On the following day, mice were intraperitoneally injected with freshly prepared RPE65-61 or vehicle (Solutol HS 15 (BASF Corp, Florham Park, NJ, USA) and dimethyl sulfoxide (DMSO) dissolved in sterile saline (0.9% NaCl)). One hour after the systemic administration of RPE65-61 or vehicle, mice were placed in a light box illuminated with white fluorescent tube lights (10,000 lux for 3 hr). The mice were returned to regular housing for 5 days and then the retinal damage was assessed.

### Example 3. HPLC Retinoid Profile Assay

Dark-adapted or light-exposed mice were sacrificed, and their eyes enucleated under dim red light. The whole eyes were homogenized with a glass grinder in lysis buffer [10 mM NH₂OH, 50% ethanol, 50% 2-(N-morpholino) ethanesulfonic acid, pH 6.5], incubated for 1 hr, and retinoids were extracted with hexane. Solvent was evaporated under argon gas, and dried retinoid samples were resuspended in 200 μl of HPLC mobile phase (11.2% ethyl acetate, 2.0% dioxane, 1.4% octanol, 85.4% hexane) and injected into HPLC (515 HPLC pump; Waters Corp., Milford, MA) with a normal phase Lichrosphere SI-60 (Alltech, Deerfield, IL) 5 μm column and isocratic mobile phase (1 ml/min) (Shin, Y. et al. 2018).

### Example 4. Histology

The superior side of the cornea was demarcated with green tattoo dye, and the eyes were carefully enucleated. The eyes were immersed in Prefer's fixative (manufactured by Anatech Ltd) for 30 min, and kept in 70% ethanol until they were embedded in paraffin. Sagittal sections along the superior-inferior retinal axis were cut, and the slides were deparaffinized prior to hematoxylin and eosin staining. Light microscopy was performed with Olympus Provis Ax-70 microscope, and the acquired images were analyzed with Image J software (NIH, Bethesda) (Rajala, A. et al. 2018).

### Example 5. Effect of Inhibitor RPE65-61 on Retinal Apoptosis

To further evaluate the effect of RPE65-61 on retinal apoptosis, retinal sections from LIRD mice were used for Terminal deoxynucleotidyltransferase dUTP nick-end labeling (TUNEL) with the In Situ Cell Death Detection Kit (Millipore/sigma Cat #:11684795910). TUNEL-positive photoreceptor cells were quantified to evaluate the effects of RPE65-61 as described (Rajala, A. et al. 2018; Ma, H. et al. 2020).

### Example 6. Optical Coherence Tomography (OCT) and Quantification of Retinal Thickness

Spectral-domain (SD)-OCT device (Bioptigen Inc. Durham. NC, USA) was used to record the retinal thickness (Qiu, F. et al. 2017; Qiu, F. et al. 2019). Images were captured with the rectangular scan at 1000 A-scans per B-scan, and 100 B-scans per frame. Total retinal thickness (TRT) was measured perpendicular to the surface of retinal pigment epithelial (RPE) layer and retinal nerve fiber layer (RNFL), 500 μm away from the center of optic nerve head (ONH) using the built-in software (InVivoVU, Bioptigen), and then averaged. The examiners were blinded to the treatment information.

### Example 7. ERG Recording

Mice were dark-adapted overnight prior to ERG recording. Mice were anesthetized by an intraperitoneal injection of 2 μl/g body weight of 40 mg/ml ketamine and 3 mg/ml xylazine diluted with saline. Pupils were dilated with 1% cyclopentolate hydrochloride ophthalmic solution (Cyclogyl) (Sandoz Inc, Princeton, NJ) and 10% phenylephrine-HCl. Hypromellose ophthalmic demulcent solution (Goniovisc; 2.5%) (HUB Pharmaceuticals LLC, Rancho Cucamonga, CA) was applied to each cornea, followed by the placement of gold wire electrodes. Stainless steel electrodes were placed into the right cheek and the tail to serve as a reference and the ground, respectively. Dark-adaptation recovery protocol was run using an Espion E3 system with a Ganzfeld ColorDome system (Diagnosys LLC, Lowell, MA) (Rajala, A. et al. 2018). The rod photoreceptor function was measured by single flash stimuli, and the cone photoreceptor function was measured (Vehicle: n=8 and RPE65-61: n=8).

### Statistical Analyses

GraphPad Prism 8.0 software (GraphPad Software, Inc., La Jolla, CA) was used for statistical analyses. Paired Student's t-test was performed to examine statistical significance (expressed as mean±SEM or SD).

### In Vitro ADME Assay Information

### Example 8. Thermodynamic Solubility Assay

Thermodynamic aqueous solubility assay in PBS (pH 7.4) was conducted by Eurofins using a shake-flask assay with a 24 h incubation period (25° C.) using HPLC-UV/VIS detection (230 nm). Aqueous solubility (μM) was determined by comparing the peak area of the principal peak in a calibration standard (200 μM) containing organic solvent (methanol/water, 60/40, v/v) with the peak area of the corresponding peak in a buffer sample. In addition, chromatographic purity (%) was defined as the peak area of the principal peak relative to the total integrated peak area in the HPLC chromatogram of the calibration standard. A chromatogram of the calibration standard of each test compound, along with a UV/VIS spectrum with labeled absorbance maxima, was generated.

### Standards for the Shake-Flask Solubility Study:

Metoprolol—192.6 μM
Rifampicin—200 μM
Ketoconazole—152.8 μM
Phenytoin—101.8 1 μM
Simvastatin—14.2 μM
Diethylstilbesterol—7.0 μM
Tamoxifen—1.9 μM

### Example 9. CYP450 Inhibition Assay

Inhibition potential (IC₅₀ values) for compounds was determined against the human cytochrome P450 (CYP) isoforms 2C9, 2C19, 2D6, and 3A4. Each recombinant human CYP isoform was tested with a standard positive and negative control, using fluorometric detection for measuring CYP activity. The measured IC₅₀ values for the respective standard inhibitors were all within expected ranges for each isoform (see below).

### IC₅₀ Concentrations of Standard CYP Inhibitors:

CYP Inhibitor IC₅₀ (μM):
2C9 Sulfaphenazole IC₅₀=3.4 μM
2C19 Tranylcypromine IC₅₀=2.8 μM
2D6 Quinidine IC₅₀=0.058 μM
3A4 Ketoconazole IC₅₀=0.0084 μM

Pre-formulated NADPH regenerating solutions, recombinant CYP isoforms 2C19 and 3A4 (Lot #3007790 and 2276593 respectively), 3-[2-(N,N-diethyl-N-methylamino)ethyl]-7-methoxy-4-methylcoumarin (AMMC), 3-cyano-7-ethoxycoumarin (CEC) and 7-benzyloxy-4-trifluoromethylcoumarin (BFC) were obtained from Corning Life Sciences (Bedford, MA). Recombinant CYP isoform 2D6 (Lot #49242) was obtained from Invitrogen (Carlsbad, CA). CYP isoform 2C9 (Lot #0446966-1) was obtained from Cayman Chemical (Ann Arbor, MI). 7-methoxy-4-trifluoromethylcoumarin (MFC), trans-2-phenylcyclopropylamine HCl (TCP), sulfaphenazole (SFZ), ketoconazole (KTZ) and quinidine (QDN) were obtained from Sigma (St. Louis, MO). All solvents and buffers were obtained from commercial sources and used without further purification.

### Methods:

Test compound was prepared as a 10 mM stock solution in acetonitrile. Four human P450 isoforms cDNA-expressed in insect cell microsomes (CYP2C9, CYP2C19, CYP2D6, and CYP3A4) were tested for inhibition by test compound using fluorescence-based assays. Nine serial dilutions (concentrations from 0-100 μM) using each test compound stock solution were prepared in black microtiter plates, in duplicate. This dilution series was incubated at 37° C. with the individual CYP isoforms and a standard fluorogenic probe substrate for each respective isoform. The concentration of the probe substrate added was at or near the Km value for each CYP isoform. Reaction mixtures contained potassium phosphate buffer, pH 7.4 and the NADPH-regenerating system. The final reaction volume was 0.20 mL and the reaction was terminated with 75 μL of stop solution (0.5 M Tris base in acetonitrile) after the appropriate incubation time (15-45 minutes). Fluorescence measurements were made at the appropriate excitation and emission wavelengths. Duplicate control wells with no test compound, duplicate blank wells containing stop solution prior to adding isoform, and a dilution series in duplicate containing a standard inhibitor for each isoform were also conducted. IC₅₀ values were calculated using a non-linear regression of the data using the four-parameter logistic model (dose response equation) fit with XLFit 5.2 from IDBS Software (Emeryville, CA), supported by linear interpolation of data points at concentrations indicating inhibition levels approximately 50% of the uninhibited rate.

### Example 10. Plasma Protein Binding Assay

Plasma protein binding (PPB) for compounds in PBS (pH 7.4) was conducted by Eurofins using equilibrium dialysis of plasma with HPLC-UV/Vis detection.

### Mean Plasma Protein Binding of Control Propranolol in Human, Rat (Sprague Dawley), Mouse (CD-1), and Dog (Beagle) Plasma

The peak areas of the test compound in the buffer and test samples were used to calculate percent binding and recovery according to the following formulas:

$$Protein binding ( \% ) = \frac{{Area}_{p} - {Area}_{b}}{{Area}_{p}} * 100$$

$$Recovery ( \% ) = \frac{{Area}_{p} + {Area}_{b}}{{Area}_{c}} * 100$$

### Where:

Areaₚ=Peak area of analyte in protein matrix
Area_{b}=Peak area of analyte in buffer
Area_{c}=Peak area of analyte in control sample

### Example 11. Metabolic Stability

### Metabolic Stability in Microsomes

Metabolic stability determinations for novel compounds and testosterone (positive control) were conducted in the presence of human, rat, mouse, and dog liver microsomes, and the results obtained. Values shown are percent of parent remaining after a 30 minute incubation. All measurements were done in duplicate. Assay results for testosterone were within an acceptable range.

### Metabolic Clearance in Microsomes

Mixed-gender human liver microsomes (Lot #1710084), male Sprague-Dawley rat liver microsomes (Lot #1610290), male CD-1 mouse liver microsomes (Lot #1710069), and male beagle dog liver microsomes (Lot #1410114) were purchased from XenoTech. The reaction mixture, minus NADPH, was prepared as described below. The test article was added into the reaction mixture at a final concentration of 1 μM. The control compound, testosterone, was run simultaneously with the test article in a separate reaction. An aliquot of the reaction mixture (without cofactor) was equilibrated in a shaking water bath at 37° C. for 3 minutes. The reaction was initiated by the addition of cofactor, and the mixture was incubated in a shaking water bath at 37° C. Aliquots (100 μL) were withdrawn at 0, 10, 20, 30, and 60 minutes. Test article and testosterone samples were immediately combined with 400 μL of ice-cold 50/50 acetonitrile (ACN)/H₂O containing 0.1% formic acid and internal standard to terminate the reaction. The samples were then mixed and centrifuged to precipitate proteins. All samples were assayed by LC-MS/MS using electrospray ionization. The peak area response ratio (PARR) to internal standard was compared to the PARR at time 0 to determine the percent remaining at each time point. Half-lives were calculated using GraphPad software, fitting to a single-phase exponential decay equation.

### Example 12. VAP-1 Stability

### Stability in Human and Dog Plasma

Emixustat was reported to undergo extensive VAP-1 metabolism in humans. Preclinical studies showed that the highest plasma deamination activity for emixustat was observed in pig and dog plasma, species that are reported to have higher concentrations of soluble VAP-1 in plasma than humans. Therefore, we compared RPE65-61 and emixustat stability in dog plasma as a robust and high throughout means by which to determine VAP-1 mediated metabolism. Studies were carried out in mixed-gender human plasma (Lot #HMN330460) and male Beagle dog plasma (Lot #BGL118112), obtained from BioIVT and collected on sodium heparin. Plasma was adjusted to pH 7.4 prior to initiating the experiments. DMSO stocks were first prepared for the test articles. Aliquots of the DMSO solutions were dosed into 1 mL of plasma, which had been pre-warmed to 37° C., at a final test article concentration of 1 μM. The vials were kept in a benchtop Thermomixer® for the duration of the experiment. Aliquots (100 μL) were taken at each time point (0, 15, 30, 60, and 120 minutes) and added to 96-well plates, which had been pre-filled with 300 μL of acetonitrile (ACN). Samples were stored at 4° C. until the end of the experiment. After the final time point was sampled, the plate was mixed and then centrifuged at 3,000 rpm for 10 minutes. Aliquots of the supernatant were removed, diluted 1:1 into distilled water, and analyzed by LC-MS/MS. The peak area response ratio to internal standard (PARR) was compared to the PARR at time 0 to determine the percent of test article remaining at each time point. Half-lives were calculated using GraphPad software, fitting to a single-phase exponential decay equation.

TABLE 1
In vitro ADME data generated for RPE65-61 and (R)-emixustat

| | [symbol] | | | | | | | | | | | |
|---|---|---|---|---|---|---|---|---|---|---|---|---|
| | [symbol] | [symbol] | | | | [symbol] | | [symbol] | | | | |
| | [symbol] | [symbol] | | | | [symbol] | | [symbol] | [symbol] | | | |
| Compound ID | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

### Chemistry

### Example 13. Preparation of 3-(Cyclohexylmethoxy)benzenethiol (4)

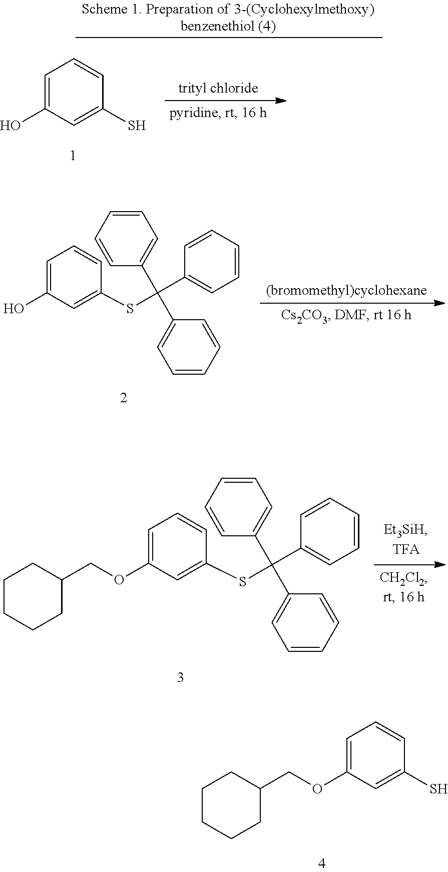 [SMILES: Oc1cccc(S)c1 ; Oc1cccc(SC(c2ccccc2)(c2ccccc2)c2ccccc2)c1 ; c1ccc(C(Sc2cccc(OCC3CCCCC3)c2)(c2ccccc2)c2ccccc2)cc1 ; Sc1cccc(OCC2CCCCC2)c1]

Step A: To a 0° C. cooled solution of 3-mercaptophenol (1) (2.00 g, 15.85 mmol) in anhydrous pyridine (20 mL) was added tritylchloride (4.60 g, 16.50 mmol). The resulting mixture was stirred at rt under an atmosphere of N₂ for 16 h. The mixture was then diluted with H₂O (30 mL) and extracted with CH₂Cl₂ (3×50 mL). The combined organic extracts were washed with brine, dried over Na₂SO₄, filtered and concentrated under reduced pressure to give crude 3-(tritylthio)phenol (2) as a brown oil (5.8 g, >99%, crude), which was taken as is into next step without purification.

Step B: To a 0° C. cooled solution of 3-(tritylthio)phenol (2, 5.8 g, 15.73 mmol) in anhydrous DMF (50 mL) were added Cs₂CO₃ (7.60 g, 23.32 mmol) and (bromomethyl)cyclohexane (3.3 g, 18.63 mmol). The resulting mixture was stirred at rt under an atmosphere of N₂ for 16 h. The mixture was then diluted with H₂O (30 mL) and extracted with EtOAc (3×50 mL). The combined organic extracts were washed with brine, dried over Na₂SO₄, filtered and concentrated under reduced pressure. The resulting crude residue was chromatographed over silica gel (0-15% EtOAc in hexanes) to give (3-(cyclohexylmethoxy)phenyl) (trityl)sulfane (3) as an oil (6.2 g, 85%, crude). The material contained an inseperable impurity, which was taken ino next step.

Step C: To a 0° C. cooled solution of (3-(cyclohexylmethoxy)phenyl) (trityl)sulfane (3, 6.2 g, 13.34 mmol) in CH₂Cl₂ (30 mL) were added TFA (15 mL, 196 mmol) and Et₃SiH (6.4 mL, 40.07 mmol). The resulting mixture was stirred at rt under an atmosphere of N₂ for 16 h. The mixture was concentrated under reduced pressure and the residue was diluted with H₂O (50 mL). The aqueous mixture was extracted with CH₂Cl₂ (3×50 mL) and the combined organic extracts were washed with brine, dried over Na₂SO₄, filtered and concentrated under reduced pressure. The resulting residue was chromatographed over silica gel (0-5% EtOAc in hexanes) to give 3-(cyclohexylmethoxy)benzenethiol (4) as a yellow oil that contained an inseparable impurity (1.5 g, 50%, crude).

### Example 14. Preparation of (±)-(2-aminoethyl) (3-(cyclohexylmethoxy)phenyl) (imino)-λ⁶-sulfanone ((±) -9; RPE65-61)

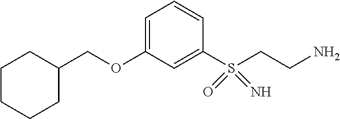 [SMILES: N=S(=O)(CCN)c1cccc(OCC2CCCCC2)c1]

Step A: To a solution of 3-(cyclohexylmethoxy)benzenethiol (4, 1.5 g, 6.74 mmol) in anhydrous DMF (7 mL) were added Cs₂CO₃ (6.60 g, 20.25 mmol) and tert-butyl (2-chloroethyl)carbamate (2.4 g, 13.36 mmol) and the resulting mixture was heated at 85° C. under an atmosphere of N₂ for 16 h. The mixture was allowed to cool to rt and then diluted with H₂O (30 mL). The aqueous mixture was extracted with EtOAc (3×50 mL) and the combined organic extracts were washed with brine, dried over Na₂SO₄, and concentrated under reduced pressure. The resulting residue was chromatographed over silica gel (0-10% EtOAc in hexanes) to give tert-butyl (2-((3-(cyclohexylmethoxy)phenyl)thio)ethyl)carbamate (5) as a colorless oil (1.1 g, 45%): ¹H NMR (400 MHz, acetone-d₆) δ 7.16 (t, J=7.6 Hz, 1H), 6.95 (s, 1H), 6.89 (d, J=7.6 Hz, 1H), 6.71 (d, J=8.4 Hz, 1H), 3.79 (d, J=6.4 Hz, 2H), 3.27-3.22 (m, 2H), 3.02-3.00 (m, 2H), 1.86-1.64 (m, 6H), 1.41 (s, 9H), 1.37-1.18 (m, 4H), 1.01-1.00 (m, 2H); ESI MS m/z 297 [M+H]⁺. ESI MS m/z 366 [M+H]⁺.

Step B: To a 0° C. solution of tert-butyl (2-((3-(cyclohexylmethoxy)phenyl)thio)ethyl)carbamate (5, 1.2 g, 3.28 mmol) in a mixture of H₂O (18 mL) and CH₃OH (12 mL) was added NaIO₄ (1.05 g, 4.91 mmol). The mixture was heated at 80° C. for 16 h, then allowed to cool to rt and concentrated under reduced pressure to remove CH₃OH. The resulting aqueous mixture was extracted with EtOAc (3×50 mL) and the combined organic extracts were washed with brine, dried over Na₂SO₄, and concentrated under reduced pressure. The resulting residue was chromatographed over silica gel (0-50% EtOAc in hexane) to give (±)-tert-butyl (2-((3-(cyclohexylmethoxy)phenyl)sulfinyl)ethyl)carbamate ((±)-6) as a white solid (0.580 g, 58%) along with recovered tert-butyl (2-((3-(cyclohexylmethoxy)phenyl)thio)ethyl)carbamate (5, 0.250 g): ¹H NMR (400 MHz, acetone-d₆) δ 7.45 (t, J=8 Hz, 1H), 7.22 (s, 1H), 7.17 (d, J=7.6 Hz, 1H), 7.04 (d, J=8.4 Hz, 1H), 3.84 (d, J=6.4 Hz, 2H), 3.49-3.30 (m, 2H), 3.12-3.06 (m, 1H), 2.89-2.83 (m, 1H), 1.86-1.64 (m, 6H), 1.42 (s, 9H), 1.36-1.19 (m, 4H), 1.17-1.04 (m, 2H), ESI MS m/z 382 [M+H]⁺.

Step C: To a 0° C. cooled solution of tert-butyl (2-((3-(cyclohexylmethoxy)phenyl)sulfinyl)ethyl)carbamate (6, 0.550 g, 1.44 mmol) in CH₂Cl₂ (30 mL) were added 2,2,2-trifluoroacetamide (0.444 g, 3.92 mmol), MgO (0.230 g, 5.71 mmol), PhI(OAc)₂ (0.695 g, 2.16 mmol) and Rh₂(OAc)₄ (31.8 mg, 0.072 mmol). The resulting mixture was stirred at rt under an atmosphere of N₂ for 16 h and was then filtered through Celite. The filtrate was concentrated under reduced pressure and the resulting residue was chromatographed over silica gel (0-60% EtOAc in hexane) to give (±)-tert-butyl (2-(3-(cyclohexylmethoxy)-N-(2,2,2-trifluoroacetyl)phenylsulfonimidoyl)ethyl)carbamate ((±)-7) as a white solid (0.600 g, 85%): ¹H NMR (400 MHz, acetone-d₆) δ 7.63 (t, J=8.4 Hz, 1H), 7.57-7.53 (m, 2H), 7.36 (d, J=8.4 Hz, 1H), 4.00-3.90 (m, 4H), 3.52-3.45 (m, 2H), 1.87-1.65 (m, 6H), 1.33 (s, 9H), 1.27-1.04 (m, 5H), ESI MS m/z 493 [M+H]⁺.

Step D: To a stirred solution of (±)-tert-butyl (2-(3-(cyclohexylmethoxy)-N-(2,2,2-trifluoroacetyl)phenylsulfonimidoyl)ethyl)carbamate as ((±)-7, 0.600 g, 1.22 mmol) in CH₃OH (10 mL) was added K₂CO₃ (0.841 g, 6.09 mmol) and the resulting mixture was stirred at rt for 12 h. The mixture was concentrated under reduced pressure and the resulting material was taken up in CH₂Cl₂ (50 mL). Separation of suspended solid matter was achieved via filtration through Celite. The filter cake was washed with CH₂Cl₂ (100 mL) and the filtrate was concentrated under reduced pressure to give crude (±)-tert-butyl (2-(3-(cyclohexylmethoxy)phenylsulfonimidoyl)ethyl)carbamate ((±)-8) as colorless oil, which was used as is in the next step (0.450 g, 93%): ESI MS m/z 397 [M+H]⁺.

Step E: To a 0° C. cooled solution of (±)-tert-butyl (2-(3-(cyclohexylmethoxy)phenylsulfonimidoyl)ethyl)carbamate ((±)-8, 0.450 g 1.13 mmol) in CH₂Cl₂ (10 mL) was added TFA (5 mL, 65.33 mmol). The mixture was stirred for 1 h at rt and was carefully neutralized via addition of a saturated aqueous solution of NaHCO₃. The biphasic mixture was separated and the aqueous layer was further extracted with CH₂Cl₂ (3×50 mL). The combined organic layers were washed with brine, dried over Na₂SO₄, filtered and concentrated under reduced pressure. The resulting residue was chromatographed over silica gel (0-10% CH₃OH in CH₂Cl₂) to give (±)-(2-aminoethyl) (3-(cyclohexylmethoxy)phenyl) (imino)-λ⁶-sulfanone ((±)-9; RPE65-61) as a colorless oil (0.130 g, 39%): ¹H NMR (400 MHz, DMSO-d₆) δ 7.50 (t, J=8 Hz, 1H), 7.42 (d, J=8 Hz, 1H), 7.34 (s, 1H), 7.20 (d, J=7.2 Hz, 1H), 3.84 (d, J=6 Hz, 2H), 3.24-3.17 (m, 2H), 2.77-2.73 (m, 2H), 2.07-1.63 (m, 6H), 1.26-1.17 (m, 3H), 1.09-1.03 (m, 2H); ¹³C NMR (500 MHz, DMSO-d₆) δ 159.5, 130.8, 120.3, 119.7, 113.9, 73.6, 37.5, 36.1, 29.6, 26.4, 25.7; ESI MS m/z 297 [M+H]⁺; HRMS (ESI⁺) C₁₅H₂₄N₂O₂S calcd [M+H]⁺=297.1631, observed [M+H]⁺=297.1621; combustion analysis (% C,H,N): calcd for C₁₅H₂₄N₂O₂S·0.7H₂O: % C=58.3; % H=8.28; % N=9.06; found: % C=58.66; % H=8.02; % N=8.7; HPLC 98.8% (AUC), t_{R}=12.4 min.

In the reaction schemes described hereinafter, the synthesis of compounds of the formula (I) are described.

### General Procedures for Preparing Amino Sulfoximine IX

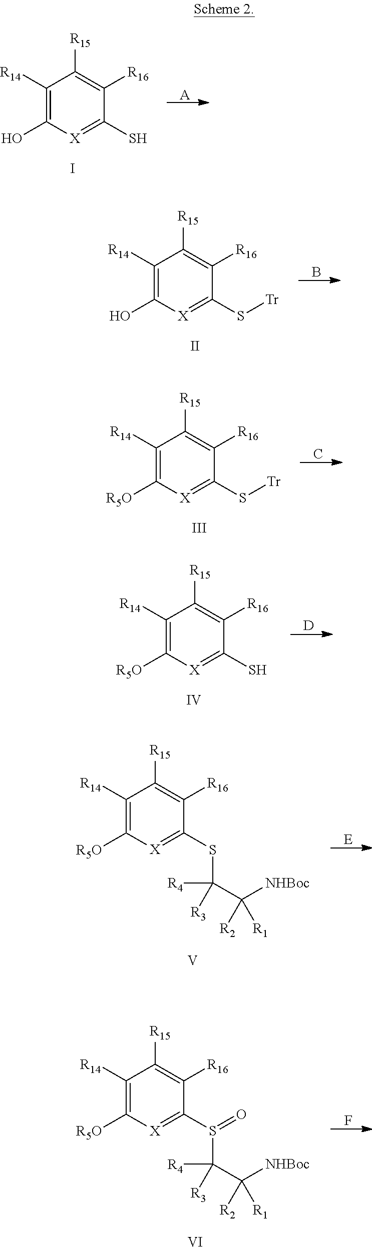

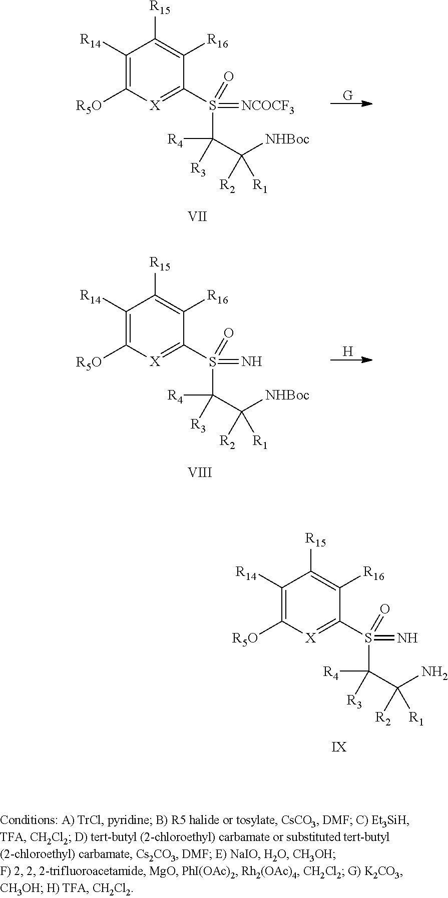

General Procedure (GP-A) for Benzenethiol Trityl Group Protection (II): Tritylchloride (TrCl) (1.05 equivalents) is slowly added to a 0° C. cooled solution of 3-mercaptophenol (1 equivalent) in anhydrous pyridine (0.25 M) and the mixture is stirred under an atmosphere of N₂ for 16 at rt. The mixture is diluted with H₂O and extracted with CH₂Cl₂. The combined organic extracts are washed with brine, dried over Na₂SO₄, filtered and concentrated under reduced pressure to afford desired trityl protected benzenethiol II.

General Procedure (GP-B) for Phenol Ether Formation (III): To a 0° C. cooled solution of 3-(tritylthio)phenol II (1 equivalent) in DMF (0.25 M) are added Cs₂CO₃ (1.5 equivalents) and R5 halide or tosylate (1.2 equivalents). The resulting mixture is stirred under an atmosphere of N₂ for 16 at rt. The mixture is diluted with H₂O and extracted with EtOAc. The combined organic extracts are washed with brine, dried over Na₂SO₄, filtered and concentrated under reduced pressure. The resulting crude residue is chromatographed over silica gel (0-15% EtOAc in hexanes) to give desired ether III.

General Procedure (GP-C) for Benzenethiol Formation (IV): To a 0° C. cooled solution of III in CH₂Cl₂ (0.25 M) are added TFA (15 equivalents) and Et₃SiH (3 equivalents). The resulting mixture is stirred under an atmosphere of N₂ for 16 at rt. The mixture is diluted with H₂O and extracted with CH₂Cl₂. The combined organic extracts are washed with brine, dried over Na₂SO₄, filtered and concentrated under reduced pressure. The resulting crude residue is chromatographed over silica gel (0-5% EtOAc in hexanes) to give 3-(cyclohexylmethoxy)benzenethiol IV.

General Procedure (GP-D) for Thioether Formation (V): To a solution of benzenethiol IV (1 equivalent) in DMF (0.25 M mL) are added Cs₂CO₃ (3 equivalents) and tert-butyl (2-chloroethyl)carbamate (2 equivalents). The resulting mixture is heated at 85° C. under an atmosphere of N₂ for 16 h. The mixture is allowed to cool to rt and is diluted with H₂O. The aqueous mixture is extracted with EtOAc and the combined organic extracts are washed with brine, dried over Na₂SO₄, filtered and concentrated under reduced pressure. The resulting crude residue is chromatographed over silica gel (0-10% EtOAc in hexanes) to give tert-butyl thioether V. The product structure was verified by ¹H NMR and mass analysis.

General Procedure (GP-E) for Sulfone Formation (VI): To a 0° C. cooled solution of thioether V (1 equivalent) in 3:2 mixture of H₂O:CH₃OH (0.25 M) is added NaIO₄ (1.5 equivalents). The resulting mixture is heated at 85° C. under an atmosphere of N₂ for 16 h. The mixture is allowed to cool to rt and then concentrated under reduced pressure. The resulting aqueous mixture is extracted with EtOAc and the combined organic extracts are washed with brine, dried over Na₂SO₄, filtered and concentrated under reduced pressure. The resulting crude residue is chromatographed over silica gel (0-50% EtOAc in hexane) to give desired sulfone VI. The product structure was verified by ¹H NMR and mass analysis.

General Procedure (GP-F) for Trifluoromethyl Substituted Sulfoximine Formation (VII): To a 0° C. cooled solution of sulfone VI (1 equivalent) in CH₂Cl₂ (0.25 M) are added 2,2,2-trifluoroacetamide (2 equivalents), MO (4 equivalents), PhI(OAc)₂ (1.5 equivalents), and Rh₂(OAc)₄ (0.05 equivalents). The mixture is stirred at rt under an atmosphere of N₂ for 16 h, then filtered through a pad of celite. The filtrate is concentrated under reduced pressure and the resulting crude residue is chromatographed over silica gel (0-60% EtOAc in hexane) to afford desired trifluoromethyl substituted sulfoximine VII. The product structure was verified by ¹H NMR and mass analysis.

General Procedure (GP-G) for Sulfoximine Deprotection (VIII): To a solution of trifluoromethyl substituted sulfoximine VII (1 equivalent) in CH₃OH (0.50 M) is added K₂CO₃ (5 equivalents) and the resulting mixture is stirred at rt for 12 h. The mixture is then concentrated under reduced pressure and the residue is diluted with CH₂Cl₂. The mixture is filtered through a pad of celite and the filtrate is concentrated under reduced pressure to afford desired deprotected sulfoximine VIII.

General Procedure (GP-H) for Amino Sulfoximine Formation (IX): To a 0° C. cooled solution of Boc-protected sulfoximine VIII (1 equivalent) in CH₂Cl₂ (0.25 mL) is added TFA (57 equivalents) and the resulting mixture is stirred at rt for 1 h. The mixture is carefully neutralized via addition of saturated aqueous NaHCO₃ solution. The biphasic mixture is separated and the aqueous layer is extracted with CH₂Cl₂. The combined organic layers are washed with brine, dried over Na₂SO₄, filtered and concentrated under reduced pressure. The resulting crude residue is chromatographed over silica gel (0-10% CH₃OH in CH₂Cl₂) to afford desired amino sulfoximine IX. The product structure was verified by ¹H NMR and mass analysis.

### General Procedures for Preparing Amino Sulfoximine XV

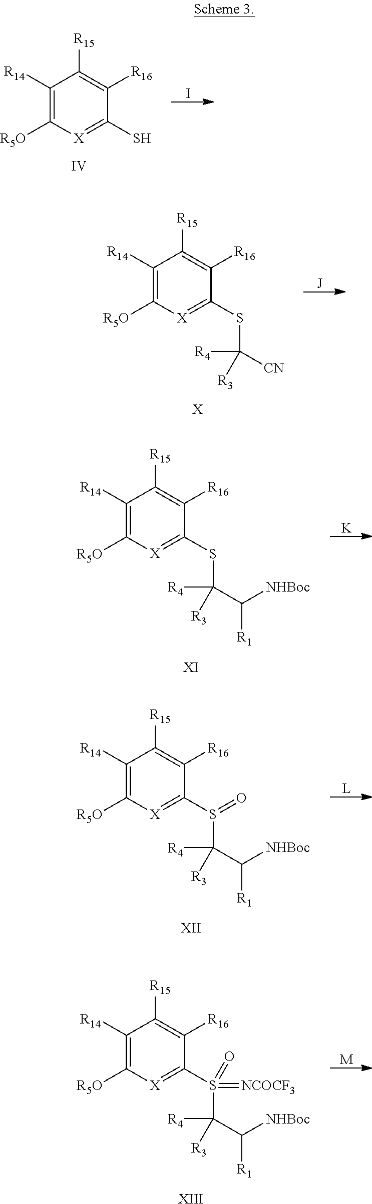

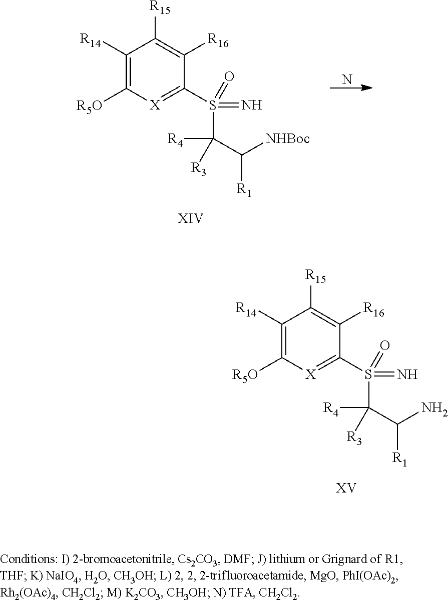

General Procedure (GP-I) for Thioether Formation (X): To a solution of benzenethiol IV (1 equivalent) in DMF (0.25 M mL) are added Cs₂CO₃ (3 equivalents) and 2-bromoacetonitrile (2 equivalents). The resulting mixture is heated at 85° C. under an atmosphere of N₂ for 16 h. The mixture is allowed to cool to rt and is diluted with H₂O. The aqueous mixture is extracted with EtOAc and the combined organic extracts are washed with brine, dried over Na₂SO₄, filtered and concentrated under reduced pressure. The resulting crude residue is chromatographed over silica gel (0-10% EtOAc in hexanes) to give tert-butyl thioether X. The product structure was verified by ¹H NMR and mass analysis.

General Procedure (GP-J) for Thioether Formation (XI): To a 0° C. cooled solution of nitrile X (1 equivalent) in THF (0.25 M mL) is added R1 lithium or Grignard reagent (1.05 equivalents). The resulting mixture is stirred at rt under an atmosphere of N₂ for 16 h. The mixture is quenched with saturated aqueous NH₄Cl solution and the aqueous mixture is extracted with EtOAc and the combined organic extracts are washed with brine, dried over Na₂SO₄, filtered and concentrated under reduced pressure. The resulting crude residue is chromatographed over silica gel (0-10% EtOAc in hexanes) to give an intermediary amine that is dissolved in CH₂Cl₂ (0.25 M). The solution is cooled to 0° C. and di-tert-butyl dicarbonyl (Boc₂O) is added (3 equivalents) and the mixture is stirred at rt for 16 h under an atmosphere of N₂. The mixture is diluted with H₂O and extracted with EtOAc. The combined organic extracts are washed with brine, dried over Na₂SO₄, filtered and concentrated under reduced pressure. The resulting crude residue is chromatographed over silica gel (0-10% EtOAc in hexanes) to afford Boc-protected thioether XI. The product structure was verified by ¹H NMR and mass analysis.

General Procedure (GP-K) for Sulfone Formation (XII): To a 0° C. cooled solution of thioether XI (1 equivalent) in 3:2 mixture of H₂O:CH₃OH (0.25 M) is added NaIO₄ (1.5 equivalents). The resulting mixture is heated at 85° C. under an atmosphere of N₂ for 16 h. The mixture is allowed to cool to rt and then concentrated under reduced pressure. The resulting aqueous mixture is extracted with EtOAc and the combined organic extracts are washed with brine, dried over Na₂SO₄, filtered and concentrated under reduced pressure. The resulting crude residue is chromatographed over silica gel (0-50% EtOAc in hexane) to give desired sulfone XII. The product structure was verified by ¹H NMR and mass analysis.

General Procedure (GP-L) for Trifluoromethyl Substituted Sulfoximine Formation (XIII): To a 0° C. cooled solution of sulfone XII (1 equivalent) in CH₂Cl₂ (0.25 M) are added 2,2,2-trifluoroacetamide (2 equivalents), MO (4 equivalents), PhI(OAc)₂ (1.5 equivalents), and Rh₂(OAc)₄ (0.05 equivalents). The mixture is stirred at rt under an atmosphere of N₂ for 16 h, then filtered through a pad of celite. The filtrate is concentrated under reduced pressure and the resulting crude residue is chromatographed over silica gel (0-60% EtOAc in hexane) to afford desired trifluoromethyl substituted sulfoximine XIII. The product structure was verified by ¹H NMR and mass analysis.

General Procedure (GP-M) for Sulfoximine Deprotection (XIV): To a solution of trifluoromethyl substituted sulfoximine XIII (1 equivalent) in CH₃OH (0.50 M) is added K₂CO₃ (5 equivalents) and the resulting mixture is stirred at rt for 12 h. The mixture is then concentrated under reduced pressure and the residue is diluted with CH₂Cl₂. The mixture is filtered through a pad of celite and the filtrate is concentrated under reduced pressure to afford desired deprotected sulfoximine XIV.

General Procedure (GP-N) for Amino Sulfoximine Formation (XV): To a 0° C. cooled solution of Boc-protected sulfoximine XIV (1 equivalent) in CH₂Cl₂ (0.25 mL) is added TFA (57 equivalents) and the resulting mixture is stirred at rt for 1 h. The mixture is carefully neutralized via addition of saturated aqueous NaHCO₃ solution. The biphasic mixture is separated and the aqueous layer is extracted with CH₂Cl₂. The combined organic layers are washed with brine, dried over Na₂SO₄, filtered and concentrated under reduced pressure. The resulting crude residue is chromatographed over silica gel (0-10% CH₃OH in CH₂Cl₂) to afford desired amino sulfoximine XV. The product structure was verified by ¹H NMR and mass analysis.

### General Procedures for Preparing Amino Sulfoximine XX

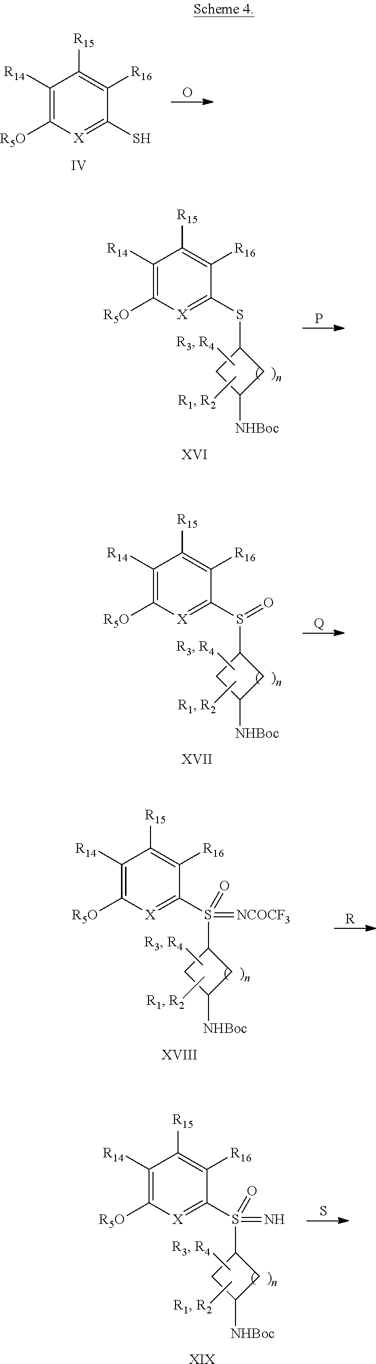

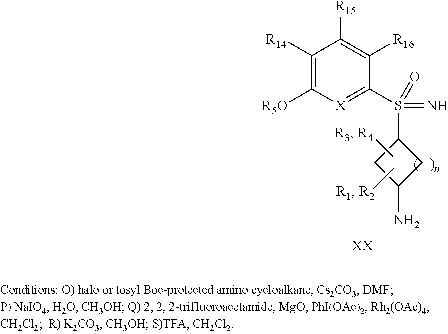

General Procedure (GP-O) for Thioether Formation (XVI): To a solution of benzenethiol IV (1 equivalent) in DMF (0.25 M mL) are added Cs₂CO₃ (3 equivalents) and halo or tosyl Boc-protected amino cycloalkane (2 equivalents). The resulting mixture is heated at 85° C. under an atmosphere of N₂ for 16 h. The mixture is allowed to cool to rt and is diluted with H₂O. The aqueous mixture is extracted with EtOAc and the combined organic extracts are washed with brine, dried over Na₂SO₄, filtered and concentrated under reduced pressure. The resulting crude residue is chromatographed over silica gel (0-10% EtOAc in hexanes) to give tert-butyl thioether XVI. The product structure was verified by ¹H NMR and mass analysis.

General Procedure (GP-P) for Sulfone Formation (XVII): To a 0° C. cooled solution of thioether XVI (1 equivalent) in 3:2 mixture of H₂O:CH₃OH (0.25 M) is added NaIO₄ (1.5 equivalents). The resulting mixture is heated at 85° C. under an atmosphere of N₂ for 16 h. The mixture is allowed to cool to rt and then concentrated under reduced pressure. The resulting aqueous mixture is extracted with EtOAc and the combined organic extracts are washed with brine, dried over Na₂SO₄, filtered and concentrated under reduced pressure. The resulting crude residue is chromatographed over silica gel (0-50% EtOAc in hexane) to give desired sulfone XVII. The product structure was verified by ¹H NMR and mass analysis.

General Procedure (GP-Q) for Trifluoromethyl Substituted Sulfoximine Formation (XVIII): To a 0° C. cooled solution of sulfone XVII (1 equivalent) in CH₂Cl₂ (0.25 M) are added 2,2,2-trifluoroacetamide (2 equivalents), MO (4 equivalents), PhI(OAc)₂ (1.5 equivalents), and Rh₂(OAc)₄ (0.05 equivalents). The mixture is stirred at rt under an atmosphere of N₂ for 16 h, then filtered through a pad of celite. The filtrate is concentrated under reduced pressure and the resulting crude residue is chromatographed over silica gel (0-60% EtOAc in hexane) to afford desired trifluoromethyl substituted sulfoximine XVIII. The product structure was verified by ¹H NMR and mass analysis.

General Procedure (GP-R) for Sulfoximine Deprotection (XIX): To a solution of trifluoromethyl substituted sulfoximine XVIII (1 equivalent) in CH₃OH (0.50 M) is added K₂CO₃ (5 equivalents) and the resulting mixture is stirred at rt for 12 h. The mixture is then concentrated under reduced pressure and the residue is diluted with CH₂Cl₂. The mixture is filtered through a pad of celite and the filtrate is concentrated under reduced pressure to afford desired deprotected sulfoximine XIX.

General Procedure (GP-S) for Amino Sulfoximine Formation (XX): To a 0° C. cooled solution of Boc-protected sulfoximine XIX (1 equivalent) in CH₂Cl₂ (0.25 mL) is added TFA (57 equivalents) and the resulting mixture is stirred at rt for 1 h. The mixture is carefully neutralized via addition of saturated aqueous NaHCO₃ solution. The biphasic mixture is separated and the aqueous layer is extracted with CH₂Cl₂. The combined organic layers are washed with brine, dried over Na₂SO₄, filtered and concentrated under reduced pressure. The resulting crude residue is chromatographed over silica gel (0-10% CH₃OH in CH₂Cl₂) to afford desired amino sulfoximine XX. The product structure was verified by ¹H NMR and mass analysis.

### General Procedures for Preparing Amino Sulfoximine XXVI

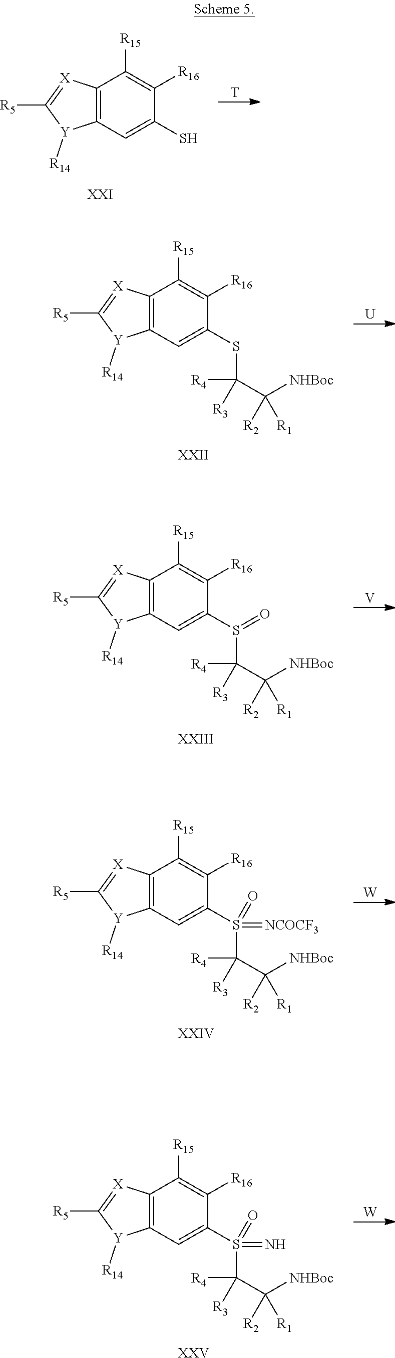

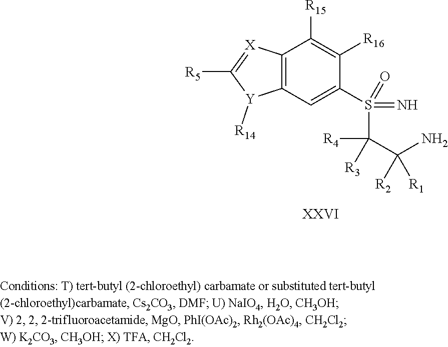

General Procedure (GP-T) for Thioether Formation (XXII): To a solution of benzenethiol XXI (1 equivalent) in DMF (0.25 M mL) are added Cs₂CO₃ (3 equivalents) and tert-butyl (2-chloroethyl)carbamate or substituted tert-butyl (2-chloroethyl)carbamate (2 equivalents). The resulting mixture is heated at 85° C. under an atmosphere of N₂ for 16 h. The mixture is allowed to cool to rt and is diluted with H₂O. The aqueous mixture is extracted with EtOAc and the combined organic extracts are washed with brine, dried over Na₂SO₄, filtered and concentrated under reduced pressure. The resulting crude residue is chromatographed over silica gel (0-10% EtOAc in hexanes) to give tert-butyl thioether XXII. The product structure was verified by ¹H NMR and mass analysis.

General Procedure (GP-U) for Sulfone Formation (XXIII): To a 0° C. cooled solution of thioether XXII (1 equivalent) in 3:2 mixture of H₂O:CH₃OH (0.25 M) is added NaIO₄ (1.5 equivalents). The resulting mixture is heated at 85° C. under an atmosphere of N₂ for 16 h. The mixture is allowed to cool to rt and then concentrated under reduced pressure. The resulting aqueous mixture is extracted with EtOAc and the combined organic extracts are washed with brine, dried over Na₂SO₄, filtered and concentrated under reduced pressure. The resulting crude residue is chromatographed over silica gel (0-50% EtOAc in hexane) to give desired sulfone XXIII. The product structure was verified by ¹H NMR and mass analysis.

General Procedure (GP-V) for Trifluoromethyl Substituted Sulfoximine Formation (XXIV): To a 0° C. cooled solution of sulfone XXIII (1 equivalent) in CH₂Cl₂ (0.25 M) are added 2,2,2-trifluoroacetamide (2 equivalents), MO (4 equivalents), PhI(OAc)₂ (1.5 equivalents), and Rh₂(OAc)₄ (0.05 equivalents). The mixture is stirred at rt under an atmosphere of N₂ for 16 h, then filtered through a pad of celite. The filtrate is concentrated under reduced pressure and the resulting crude residue is chromatographed over silica gel (0-60% EtOAc in hexane) to afford desired trifluoromethyl substituted sulfoximine XXIV. The product structure was verified by ¹H NMR and mass analysis.

General Procedure (GP-W) for Sulfoximine Deprotection (XXV): To a solution of trifluoromethyl substituted sulfoximine XXIV (1 equivalent) in CH₃OH (0.50 M) is added K₂CO₃ (5 equivalents) and the resulting mixture is stirred at rt for 12 h. The mixture is then concentrated under reduced pressure and the residue is diluted with CH₂Cl₂. The mixture is filtered through a pad of celite and the filtrate is concentrated under reduced pressure to afford desired deprotected sulfoximine XXV.

General Procedure (GP-X) for Amino Sulfoximine Formation (XXVI): To a 0° C. cooled solution of Boc-protected sulfoximine XXV (1 equivalent) in CH₂Cl₂ (0.25 mL) is added TFA (57 equivalents) and the resulting mixture is stirred at rt for 1 h. The mixture is carefully neutralized via addition of saturated aqueous NaHCO₃ solution. The biphasic mixture is separated and the aqueous layer is extracted with CH₂Cl₂. The combined organic layers are washed with brine, dried over Na₂SO₄, filtered and concentrated under reduced pressure. The resulting crude residue is chromatographed over silica gel (0-10% CH₃OH in CH₂Cl₂) to afford desired amino sulfoximine XXVI. The product structure was verified by ¹H NMR and mass analysis.

### General Procedures for Preparing Amino Sulfoximine XXXV

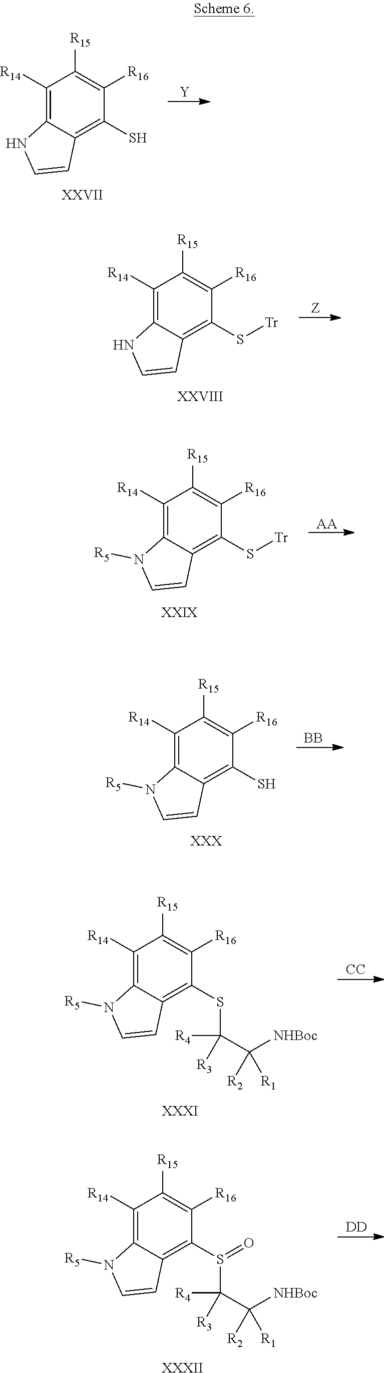

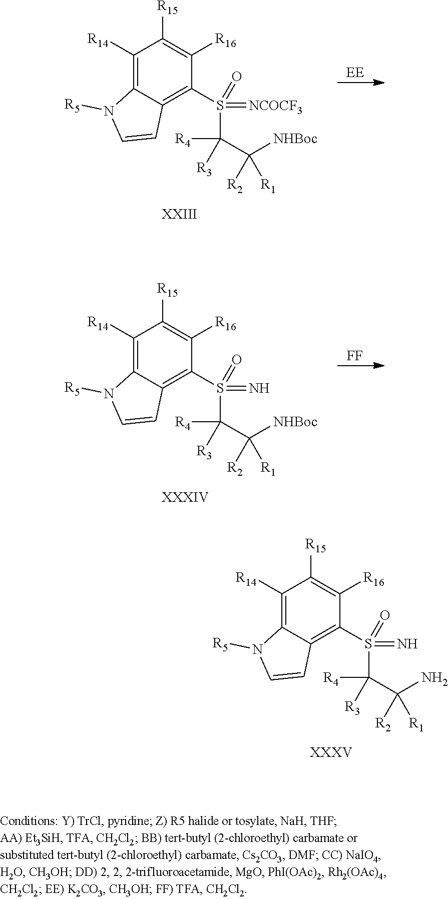

General Procedure (GP-Y) for Indole Thiol Trityl Group Protection (XXVIII): Tritylchloride (TrCl) (1.05 equivalents) is slowly added to a 0° C. cooled solution of indole thiol (1 equivalent) in anhydrous pyridine (0.25 M) and the mixture is stirred under an atmosphere of N₂ for 16 at rt. The mixture is diluted with H₂O and extracted with CH₂Cl₂. The combined organic extracts are washed with brine, dried over Na₂SO₄, filtered and concentrated under reduced pressure to afford desired trityl protected benzenethiol XXVIII.

General Procedure (GP-Z) for Indole Alkylation (XXIX): To a 0° C. cooled solution of indole XXVIII (1 equivalent) in THF (0.25 M mL) are added NaH (3 equivalents) and R5 halide or tosylate (2 equivalents). The resulting mixture is stirred at rt under an atmosphere of N₂ for 16 h. The mixture is allowed to cool to rt and is diluted with H₂O. The aqueous mixture is extracted with EtOAc and the combined organic extracts are washed with brine, dried over Na₂SO₄, filtered and concentrated under reduced pressure. The resulting crude residue is chromatographed over silica gel (0-10% EtOAc in hexanes) to give tert-butyl thioether XXIX.

The product structure was verified by ¹H NMR and mass analysis. General Procedure (GP-AA) for Indole Thiol Formation (XXX): To a 0° C. cooled solution of XXIX in CH₂Cl₂ (0.25 M) are added TFA (15 equivalents) and Et₃SiH (3 equivalents). The resulting mixture is stirred under an atmosphere of N₂ for 16 h at rt. The mixture is diluted with H₂O and extracted with CH₂Cl₂. The combined organic extracts are washed with brine, dried over Na₂SO₄, filtered and concentrated under reduced pressure. The resulting crude residue is chromatographed over silica gel (0-5% EtOAc in hexanes) to give thiol XXX. The product structure was verified by ¹H NMR and mass analysis.

General Procedure (GP-BB) for Thioether Formation (XXXI): To a solution of thiol XXX (1 equivalent) in DMF (0.25 M mL) are added Cs₂CO₃ (3 equivalents) and tert-butyl (2-chloroethyl)carbamate (2 equivalents). The resulting mixture is heated at 85° C. under an atmosphere of N₂ for 16 h. The mixture is allowed to cool to rt and is diluted with H₂O. The aqueous mixture is extracted with EtOAc and the combined organic extracts are washed with brine, dried over Na₂SO₄, filtered and concentrated under reduced pressure. The resulting crude residue is chromatographed over silica gel (0-10% EtOAc in hexanes) to give thioether XXXI. The product structure was verified by ¹H NMR and mass analysis.

General Procedure (GP-CC) for Sulfone Formation (XXXII): To a 0° C. cooled solution of thioether XXXI (1 equivalent) in 3:2 mixture of H₂O:CH₃OH (0.25 M) is added NaIO₄ (1.5 equivalents). The resulting mixture is heated at 85° C. under an atmosphere of N₂ for 16 h. The mixture is allowed to cool to rt and then concentrated under reduced pressure. The resulting aqueous mixture is extracted with EtOAc and the combined organic extracts are washed with brine, dried over Na₂SO₄, filtered and concentrated under reduced pressure. The resulting crude residue is chromatographed over silica gel (0-50% EtOAc in hexane) to give desired sulfone XXXII. The product structure was verified by ¹H NMR and mass analysis.

General Procedure (GP-DD) for Trifluoromethyl Substituted Sulfoximine Formation (XXXIII): To a 0° C. cooled solution of sulfone XXXII (1 equivalent) in CH₂Cl₂ (0.25 M) are added 2,2,2-trifluoroacetamide (2 equivalents), MO (4 equivalents), PhI(OAc)₂ (1.5 equivalents), and Rh₂(OAc)₄ (0.05 equivalents). The mixture is stirred at rt under an atmosphere of N₂ for 16 h, then filtered through a pad of celite. The filtrate is concentrated under reduced pressure and the resulting crude residue is chromatographed over silica gel (0-60% EtOAc in hexane) to afford desired trifluoromethyl substituted sulfoximine XXXIII. The product structure was verified by ¹H NMR and mass analysis.

General Procedure (GP-EE) for Sulfoximine Deprotection (XXXIV): To a solution of trifluoromethyl substituted sulfoximine XXXIII (1 equivalent) in CH₃OH (0.50 M) is added K₂CO₃ (5 equivalents) and the resulting mixture is stirred at rt for 12 h. The mixture is then concentrated under reduced pressure and the residue is diluted with CH₂Cl₂. The mixture is filtered through a pad of celite and the filtrate is concentrated under reduced pressure to afford desired deprotected sulfoximine XXXIV.

General Procedure (GP-FF) for Amino Sulfoximine Formation (XXXV): To a 0° C. cooled solution of Boc-protected sulfoximine XXXIV (1 equivalent) in CH₂Cl₂ (0.25 mL) is added TFA (57 equivalents) and the resulting mixture is stirred at rt for 1 h. The mixture is carefully neutralized via addition of saturated aqueous NaHCO₃ solution. The biphasic mixture is separated and the aqueous layer is extracted with CH₂Cl₂. The combined organic layers are washed with brine, dried over Na₂SO₄, filtered and concentrated under reduced pressure. The resulting crude residue is chromatographed over silica gel (0-10% CH₃OH in CH₂Cl₂) to afford desired amino sulfoximine XXXV. The product structure was verified by ¹H NMR and mass analysis.

### DISCUSSION

The development of safe and effective small-molecule therapeutics for blinding retinal degenerative diseases remains a major challenge. The present disclosure demonstrates that RPE65-61, RPE65-76, RPE65-77, and RPE65-78, which are novel non-retinoid compounds, effectively inhibit RPE65, a key enzyme of the visual cycle enzyme. Further, these compounds act as aldehyde traps, and RPE65-61 also protects retina structure and function from LIRD, suggesting a therapeutic potential for retinal degeneration.

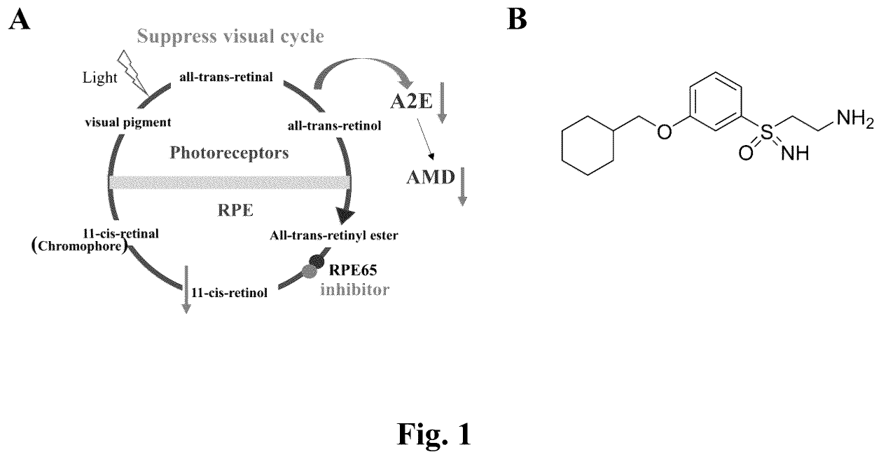

The visual cycle involves a series of biochemical reactions initiated by the interaction of a photon of light with the visual pigment protein rhodopsin, leading to an electrophysiological signal and resulting in visual perception (FIG. 1A). The process continues with a series of reactions resulting in regeneration of the rhodopsin molecule. Regeneration of the rhodopsin begins with the synthesis of 11-cis-retinal, the chromophore, and involves many retinoid metabolizing enzymes and retinoid-binding proteins. Both clinical studies and experimental animal models of retina diseases suggest that to ensure retinal health it is essential to efficiently clear all-trans-retinal, a toxic byproduct of the visual cycle, from the retina. It has been shown that the loss of clearance mechanism due to mutations in ABCA4, a gene that encodes ATP-binding cassette transmembrane protein, which removes all-trans-RAL out from rod photoreceptors, causes retina degeneration in STGD. All-trans-retinal is toxic itself and also can react with membrane lipids to form toxic bis-retinoids. Studies using Abca4^{-/-} and/or Rdh8^{-/-} mice demonstrated functional declines of retinas with corresponding retinal degeneration, which is associated with an abnormal accumulation of bis-retinoids such as A2E (Radu, R. A. et al. 2008; Maeda, A. et al. 2009 A). All-trans-retinal clearance delay, and, consequentially, abnormal accumulation of bis-retinoids, may also be a result of aging, and might cause dry AMD. It has been demonstrated that abnormal accumulation of bisretinoid condensation products has multiple disease implications for both retina and the RPE, including DNA damage, oxidative stress, complement activation, and mitochondrial dysfunction (Liang, F. Q. & Godley, B. F. 2003; Hanus, J. et al. 2015; Kaarniranta, K. et al. 2018).

One of the critical biochemical steps in the visual cycle is the conversion of all-trans-retinyl ester to 11-cis-retinol by RPE65 (FIG. 1A). RPE65 is an ideal modulator because it is a key and rate-limiting enzyme of the visual cycle and is almost exclusively expressed in the RPE (Redmond, T. M. et al. 1998; Hamel, C. P. et al. 1993; Kiser, P. D. et al. 2017; Marlhens, F. et al. 1998). A non-retinoid RPE65 inhibitor emixustat has demonstrated a dose-dependent reduction of A2E levels in Abca4^{-/-} mice after 3 months of oral administration (Bavik, C. et al. 2015). Furthermore, Rpe65^{-/-} mice, which do not contain retinoids in photoreceptors, are resistant to LIRD, as are the mice and rats treated with small-molecule inhibitors of RPE65. Hence, the visual cycle modulation provides valuable insights into developing potential therapeutic application for retinal degeneration. Conceptually, inhibition of the visual cycle can experimentally prevent retinal degeneration through reducing all-trans-retinal and/or its lipid condensation product toxicity. Partial blockade of the visual cycle would slow down rhodopsin regeneration through the visual cycle and, in turn, lower the production of toxic all-trans-retinal and its conjugation product A2E.

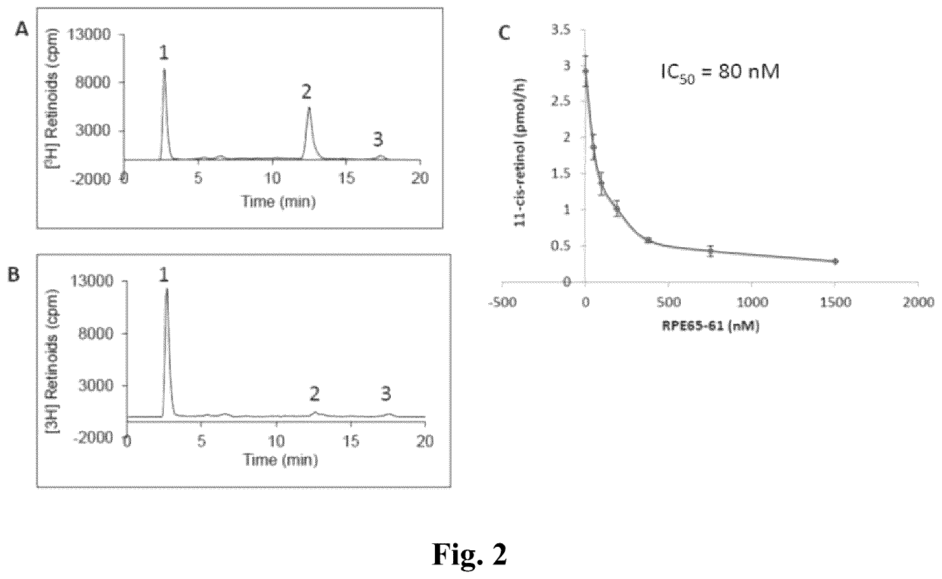

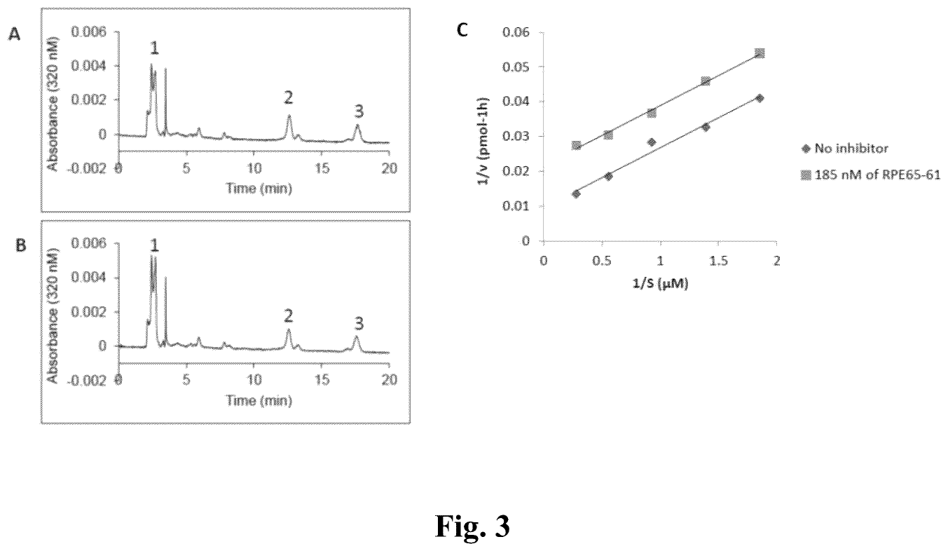

Herein we show that novel non-retinoid compounds such as RPE65-61 (FIG. 1B) efficiently and selectively inhibited RPE65 catalyzed reaction in vitro (FIGS. 2A-C). RPE65-61 inhibited RPE65 isomerase activity with an apparent IC₅₀=80 nM (FIG. 2C). RPE65-61 can bind RPE65 at its active site, competing with binding of retinyl ester substrate, or alternatively, it may bind to the enzyme-substrate complex causing uncompetitive inhibition. In order to discriminate between these two alternatives of RPE65-61 inhibition, a liposome-based isomerase assay with recombinant RPE65 was employed. RPE65-61 inhibited isomerase reaction uncompetitively suggesting that this inhibitor does not bind to the free RPE65, and it rather binds to the enzyme-substrate complex (FIGS. 3A-C) (Fersht, A. 1985). Therefore, RPE65-61 is an uncompetitive inhibitor of RPE65 with a Ki=150 nM (FIG. 3C).

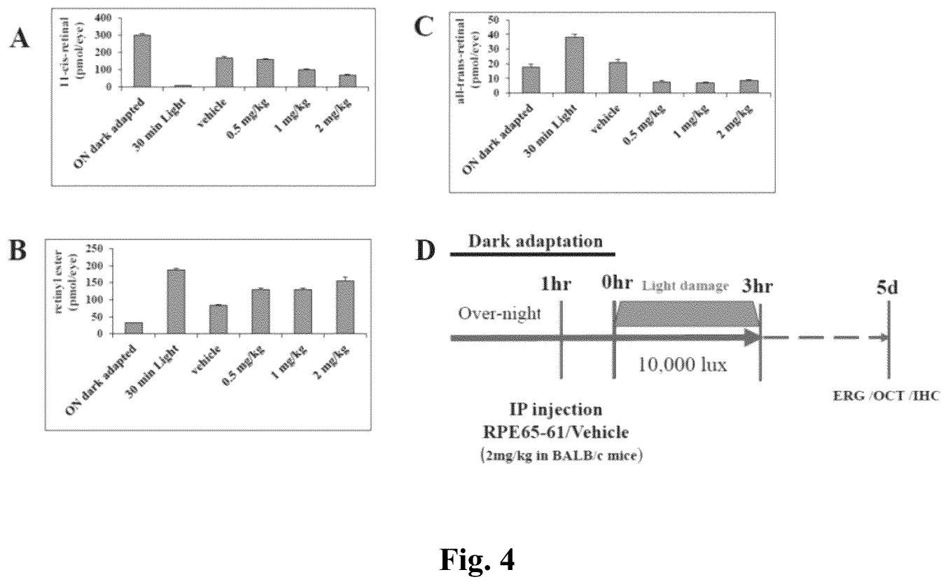

The ability of RPE65-61 to inhibit RPE65 in vivo was also investigated by determining chromophore regeneration after bleaching with 5,000 lux fluorescent light for 30 min. (FIG. 4). As shown by the HPLC retinoid profile analysis, mice injected with RPE65-61 (0.5, 1, or 2 mg/kg) showed delayed chromophore regeneration after photobleach in a dose-dependent manner, suggesting a slowed visual cycle (FIG. 4).

RPE65-61 inhibited 11-cis-retinal regeneration after photobleaching with a higher potency than another non-retinoid CU-239 described previously (FIG. 4A) (Shin, Y. et al. 2018). This effect correlates with significantly lower IC₅₀ for RPE65-61 as compared to CU239 in in vitro RPE65 inhibition. As expected, mice treated with RPE65-61 accumulate higher levels of retinyl esters because retinyl esters are converted to 11-cis-retinol more slowly in the presence of the inhibitor (FIG. 4B).

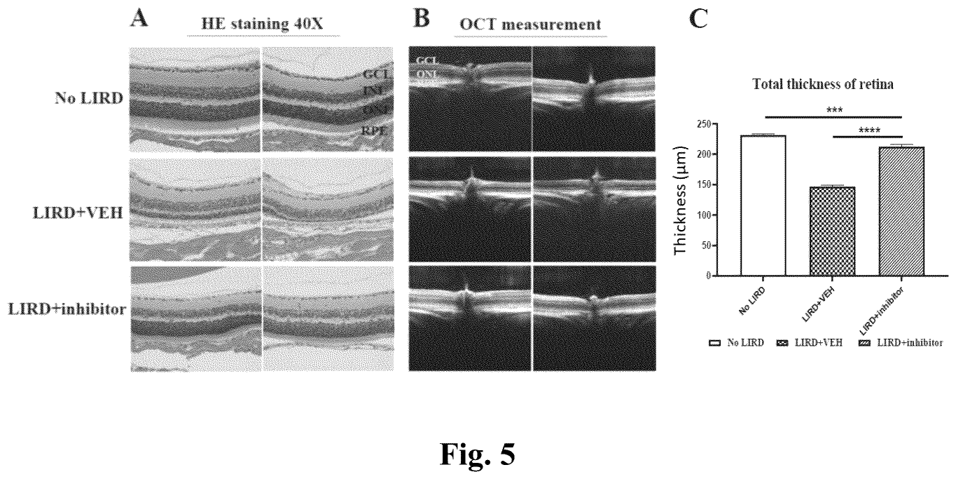

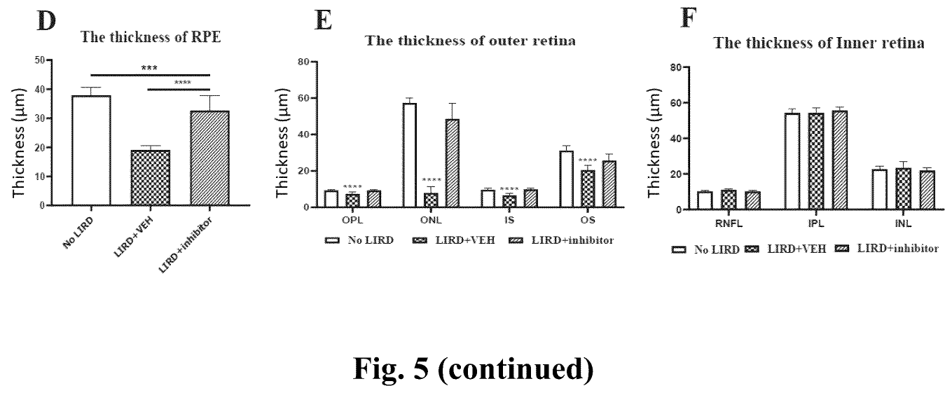

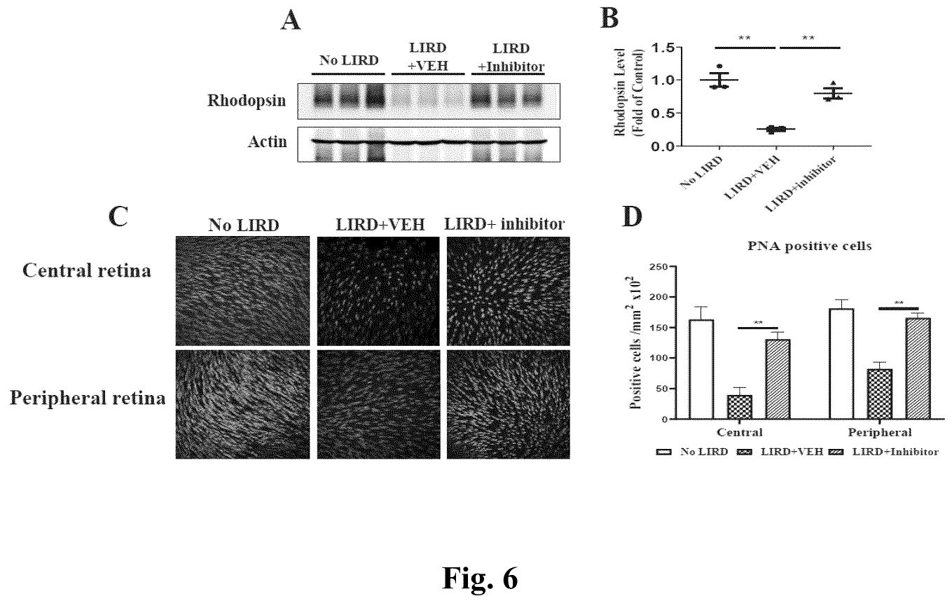

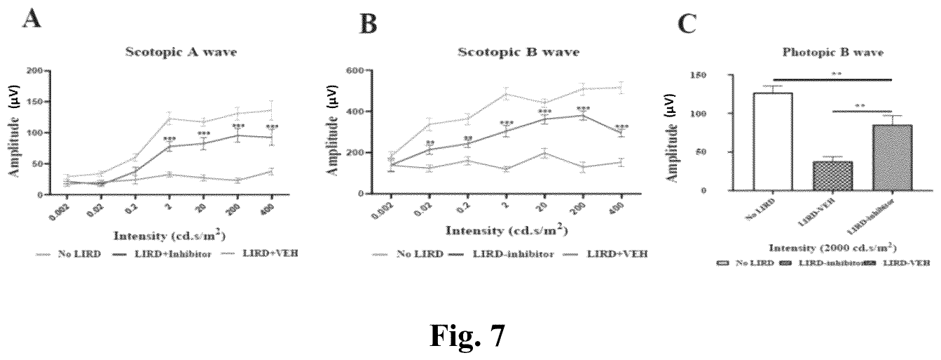

Mice were administered 2 mg/kg of RPE65-61 in order to examine whether RPE65-61-mediated visual cycle inhibition could protect the retina from light-induced retinal damage (LIRD). As expected, our post-LIRD histological analyses and functional analysis of the retina with electroretinography (ERG) demonstrated a protective effect of RPE65-61 against LIRD (FIGS. 5-7).

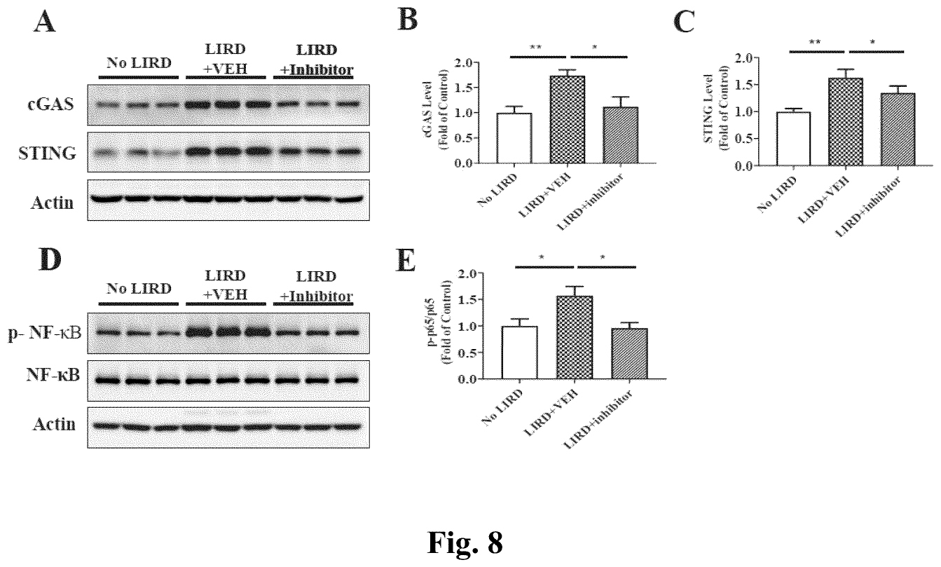

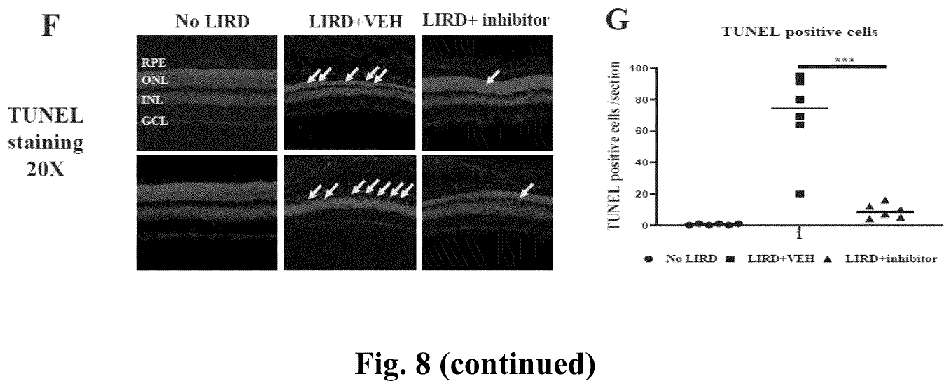

The mechanisms of retina protection from light damage through RPE65 inhibition are currently unclear. It is known that intense light causes DNA breaks (Chen, P. et al. 2019) and all-trans-retinal mediated this process (Sawada, O. et al. 2014). It has been reported that cGAS-cGAMP-STING is a sensor of DNA damage (Wu, Y. et al. 2019). This work shows that RPE65-61 inhibition protected RPE and photoreceptor cells from the activation of cGAS/STING signal pathway, possibly through the inhibition of visual cycle and, consequently, through decrease of DNA light damage, in an acute mouse model of AMD, accompanied by suppressing the upregulation of the genes involved in apoptosis and inflammatory responses (FIG. 8).

### Elimination of Undesired Route of Systemic Metabolism

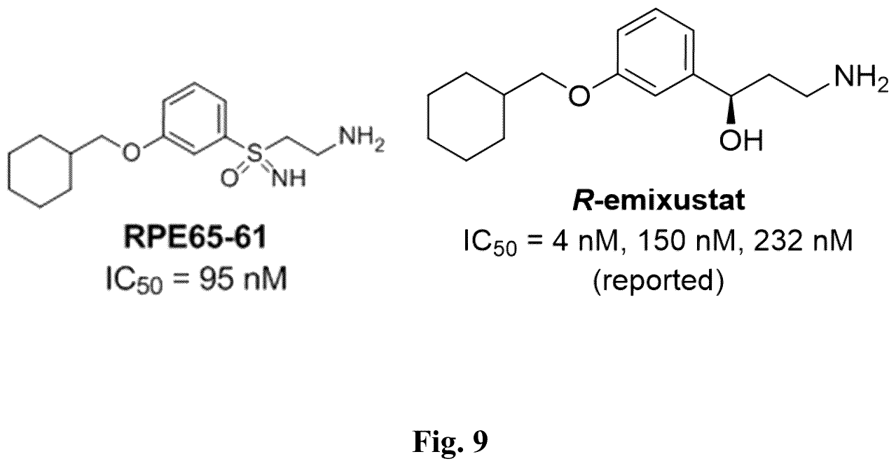

Emixustat clearance in humans relies on an unusual route of metabolism driven by oxidative deamination by Vascular Adhesion Protein-1 (VAP-1), a circulating and membrane-bound amine oxidase (Reid, M. J. et al. 2019). Emixustat (shown in FIG. 9) acts as a weak inhibitor of its metabolizing enzyme, VAP-1 (Reid, M. J. et al. 2019), which raises the drug-drug interaction (DDI) concern for therapeutics such as amlodipine that are metabolized through a similar route. Primary amine substrates that inhibit VAP-1 act as irreversible inhibitors that covalently bind to the active site (Bligt-Linden, E. et al. 2013). Given that emixustat is a VAP-1 inhibitor, this drug is likely to bind covalently to the enzyme. Covalent inhibitors generate immunogenic protein adducts which can induce a potentially deadly idiosyncratic response (Johnson, D. S. et al. 2010). Thus, unconventional route of systemic metabolism by VAP-1 and VAP-1 inhibition are negative attributes. Previous reports state that plasma from certain animal species contain high levels of VAP-1-mediated deamination activity. The highest plasma deamination activity for emixustat is seen in pig and dog plasma. Thus, a metabolic stability readout in dog plasma can provide a high throughput tool to assess unique VAP-1 liabilities.

As shown in Table 2 below, metabolic stability of both emixustat and RPE65-61 is very low in dog plasma.

TABLE 2
Metabolic stability of RPE65-61 and (R)-emixustat in human
and dog plasma. Low stability for both compounds in dog
plasma indicates extensive metabolism mediated by VAP-1.

VAP-1 Stability
(plasma half-life, min)

| Compound ID | Human | Dog |
|---|---|---|
| (R)-emixustat | >120 | 52.9 |
| RPE65-61 | >120 | 35.3 |

This indicates that RPE65-61 undergoes extensive VAP-1 oxidative metabolism, similarly to emixustat.

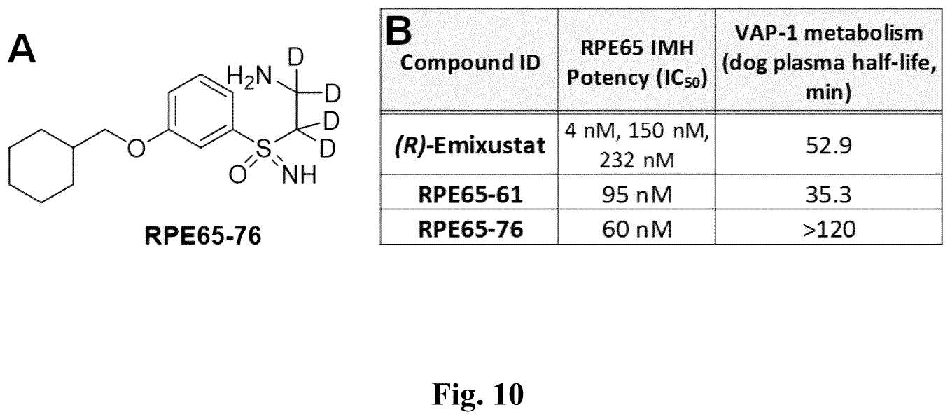

On the other hand, RPE65-76, a deuterated RPE65-61 analogue (shown in FIG. 10A), which is highly potent in the primary IMH assay (IC₅₀=60 nM), blocked VAP-1 metabolism as indicated by increased dog plasma stability for RPE65-76 (FIG. 10B). These results demonstrate that RPE65-76 has improved drug-like characteristics in comparison to emixustat and RPE65-61.

### Secondary Aldehyde Trapping Activity in RPE65 Inhibitors as a Means to Reducing Mechanism-Based Ocular Toxicity

RPE65 is an optimal and a well-validated drug target for the inhibition of the pathologic lipofuscin bisretinoid synthesis in STGD1 in dry AMD. However, pharmacological inhibition of RPE65 leads to a significant inhibition in the rate of the visual cycle. Inhibition of the visual cycle (VC) and concomitant reduction in the amount of the visual chromophore (11-cis-retinaldehyde) may produce three types of mechanism-based adverse events (“AEs”). (1) VC inhibition leads to a delay in rod function recovery from the photobleach (Maiti, P. et al. 2006; Maeda, A. et al. 2006; Golczak, M. et al. 2008; Radu, R. A. et al. 2003; Sieving, P. A. et al. 2001), which manifests in humans as delayed dark adaptation (difficulties in seeing after moving from a bright environment to a darker one). (2) Reduction in the visual chromophore in rods leads to nyctalopia (night blindness), impaired vision in dim light due to abnormal phototransduction mechanism in rods. (3) Reduction in the visual chromophore in cones leads to chromatopsia, aberrations in cone-mediated color vision. These AEs are dose dependent in humans as shown by the emixustat clinical experience (Kubota, R. et al. 2014; Kubota, R. et al. 2012; Mata, N. L. et al. 2013; Rosenfeld, P. J. et al. 2018; Dugel, P. U. et al. 2015). Direct RPE65 inhibition as a therapeutic strategy may be complicated by the severity of mechanism-based AEs (Kiser, P. D. et al. 2017).

In order to reduce levels of RPE65 inhibition below the threshold associated with AEs while still maintaining bisretinoid-lowering efficacy, the present inventors searched for RPE65 inhibitors with the ability to reduce bisretinoid synthesis through an additional secondary mechanism: retinaldehyde trapping. Retinaldehydes are direct bisretinoid precursors, and it was proposed that their neutralization through the formation of reversible Schiff base adducts using primary amine-containing drugs may reduce biosynthesis of lipofuscin bisretinoids and ameliorate direct aldehyde toxicity in the retina (Kiser, P. D. et al. 2017; Maeda, A. et al. 2012; Maeda, A. et al. 2009 B; Palczewski, K. et al. 2010). The reduction of all-trans-retinal to all-trans-retinol in rods is a relatively slow process (Rozanowska, M. et al. 2005), which is even slower in STGD1 patients with ABCA4 mutations. Formation of Schiff base conjugates is reversible, but even a temporary and reversible aldehyde neutralization can facilitate all-trans-retinal clearance and decrease bisretinoid synthesis by reducing peak levels of free all-trans-retinal that is released from opsin following the exposure to intense light. A set of the FDA-approved drugs containing primary amines were tested in the Abca4^{-/-}/Rdh8^{-/-} mouse model with a significant 40% reduction in bisretinoid content shown for some compounds (Maeda, A. et al. 2012). A reported isopropyl emixustat derivative that was significantly devoid of RPE65 inhibitory activity was still capable of forming Schiff base adducts with retinaldehydes and protect the retina from retinaldehyde-mediated damage without visual function suppression typical of RPE65 inhibitors (Kiser, P. D. et al. 2017). Furthermore, a recently reported series of peptide derivatives of retinylamine that were inactive at RPE65 demonstrated efficacy against light-induced retinal degeneration in Abca4^{-/-}Rdh8^{-/-} mice (Yu, G. et al. 2021).

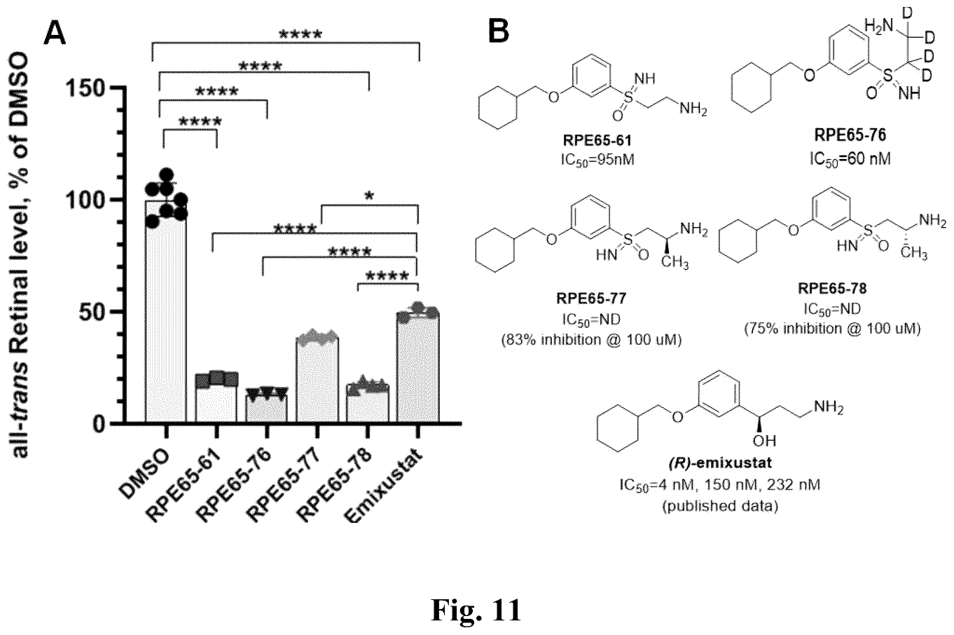

To design agents with optimal balance of RPE65 inhibition and retinaldehyde sequestration activity, and to establish the ability of compounds from RPE65-61 and its analogues to act as retinaldehyde traps, the ability of these compounds to inactivate all-trans-retinal (atRAL) in vitro was assessed. After 30 min incubation of compounds with atRAL in conditions favoring formation of Schiff base (absolute ethanol with 1.5% acetic acid in the presence of anhydrous MgSO₄), the amount of remaining atRAL was assessed spectrophotometrically. As shown in FIG. 11, all tested compounds were able to neutralize atRAL, reducing the amount of atRAL in the reaction mix. The extent of atRAL reduction for emixustat was 50% while RPE65-61, its deuterated analogue RPE65-76, and RPE65-78 were more potent showing 80%, 87%, and 83% atRAL reduction, respectively. The difference in atRAL inactivation between emixustat and the compounds of the present disclosure was statistically significant (FIG. 11A). RPE65-61 and RPE65-76 combine excellent potency in the primary RPE65 inhibition assay (IC₅₀ of 95 nM and 60 nM, respectively) with very good retinaldehyde neutralization ability in the in vitro assay. Moreover, as shown above, RPE65-76 is not susceptible to oxidative metabolism by VAP-1, demonstrating a significant improvement in drug-like characteristics over emixustat and RPE65-61. Furthermore, the methylated analogues RPE65-77 and RPE65-78 are also likely to avoid extensive VAP-1 mediated oxidation via steric shielding of the metabolic hot spot. Thus, a set of new optimized bispecific compounds that can combine RPE65 inhibition with atRAL inactivation have been identified.

The compounds described herein are novel RPE65 inhibitors that contain a structurally distinct sulfoximine group. The presence of this group distinguishes these compounds from known RPE65 inhibitors and is predicted to confer certain advantages since it can modulate the pKa of the amine in the beta-position (Mäder, P. & Kattner, L 2020; Frings, M. et al. 2017; Lücking, U. 2019). In fact, it is predicted that the pKa of the beta amine would be approximately 8, which is significantly lower than the 9-10 predicted for the corresponding —OH of emixustat. The lower pKa of the compounds of the present disclosure conferred by the presence of the sulfoximine group can potentially be translated to better permeability, oral bioavailability, and to better accumulation into the retina, since such compounds would be less ionized. Additionally, since it is known that basic amines can get trapped into lysosomes, the ability of the sulfoximine group to lower the pKa could potentially overcome the lysosomal storage liability exhibited by emixustat.

In addition to Stargardt disease, RPE65 inhibitors are used for the treatment of other forms of macular degeneration characterized by accumulation of lipofuscin, such as dry AMD and Best disease, which are currently untreatable. Despite the unmet medical need, there is no therapy for Stargardt's disease. No clinically validated drug targets for STGD treatment have been identified in recent decades. Non-retinoid RPE65 antagonists identified herein represent novel structural series for the treatment of the diseases described above.

### REFERENCES

Allikmets, R. et al. (1997) A photoreceptor cell-specific ATP-binding transporter gene (ABCR) is mutated in recessive Stargardt macular dystrophy. Nature genetics 15, 236-246.
Bavik, C. et al. (2015) Visual Cycle Modulation as an Approach toward Preservation of Retinal Integrity. PLoS One 10, e0124940.
Bergmann, M. (2004) Inhibition of the ATP-driven proton pump in RPE lysosomes by the major lipofuscin fluorophore A2-E may contribute to the pathogenesis of age-related macular degeneration. FASEB J 18, 562-564.
Birnbach, C. D. et al. (1994) Histopathology and immunocytochemistry of the neurosensory retina in fundus flavimaculatus. Ophthalmology 101, 1211-1219.
Bligt-Linden, E. et al. (2013) Novel pyridazinone inhibitors for vascular adhesion protein-1 (VAP-1): old target-new inhibition mode. J Med Chem 56, 9837-9848.
Boyer, N. P. et al. (2012) Lipofuscin and N-retinylidene-N-retinylethanolamine (A2E) accumulate in retinal pigment epithelium in absence of light exposure: their origin is 11-cis-retinal. J Biol Chem 287, 22276-22286.
Brandstetter, C. et al. (2015) Complement Component C5a Primes Retinal Pigment Epithelial Cells for Inflammasome Activation by Lipofuscin-mediated Photooxidative Damage. J Biol Chem 290, 31189-31198.
Brandstetter, C. et al. (2015) Light induces NLRP3 inflammasome activation in retinal pigment epithelial cells via lipofuscin-mediated photooxidative damage. J Mol Med 93, 905-916.
Cavallotti CAPaC, L. (2008) Age-related changes of the human eye. Humana Press.
Chen, P. et al. (2019) Retinal Neuron Is More Sensitive to Blue Light-Induced Damage than Glia Cell Due to DNA Double-Strand Breaks. Cells 8, 68.
Cruickshanks, K. J. et al. (1997) The prevalence of age-related maculopathy by geographic region and ethnicity. The Colorado-Wisconsin Study of Age-Related Maculopathy. Archives of ophthalmology (Chicago, Ill: 1960) 115, 242-250.
De Laey, J. J. & Verougstraete, C. (1995) Hyperlipofuscinosis and subretinal fibrosis in Stargardt's disease. Retina 15, 399-406.
Dorey, C. K. et al. (1989) Cell loss in the aging retina. Relationship to lipofuscin accumulation and macular degeneration. Investigative ophthalmology & visual science 30, 1691-1699.
Dugel, P. U. et al. (2015) Phase ii, randomized, placebo-controlled, 90-day study of emixustat hydrochloride in geographic atrophy associated with dry age-related macular degeneration. Retina 35, 1173-1183.
Fersht, A. Enzyme structure and mechanism. (WH Freeman & Co, 1985).
Frings, M. et al. (2017) Sulfoximines from a Medicinal Chemist's Perspective: Physicochemical and in vitro Parameters Relevant for Drug Discovery. Eur J Med Chem 126, 225-245.
Gollapalli, D. R. & Rando, R .R. (2004) The specific binding of retinoic acid to RPE65 and approaches to the treatment of macular degeneration. Proc. Natl. Acad. Sci. USA 101, 10030-10035.
Golczak, M. et al. (2008) Metabolic basis of visual cycle inhibition by retinoid and nonretinoid compounds in the vertebrate retina. J Biol Chem 283, 9543-9554.
Hamel, C. P. et al. (1993) Molecular cloning and expression of RPE65, a novel retinal pigment epithelium-specific microsomal protein that is post-transcriptionally regulated in vitro. J Biol Chem 268, 15751-15757.
Hanus, J. et al. (2015) RPE necroptosis in response to oxidative stress and in AMD. Ageing Res Rev 24 (Pt B), 286-298.
Jaakson, K. et al. (2003) Genotyping microarray (gene chip) for the ABCR (ABCA4) gene. Human mutation 22, 395-403.
Jin, M. et al. (2005) Rpe65 is the retinoid isomerase in bovine retinal pigment epithelium. Cell 122, 449-459.
Johnson, D. S. et al. (2010) Strategies for discovering and derisking covalent, irreversible enzyme inhibitors. Future Med Chem 2, 949-964.
Kaarniranta, K. et al. (2018) PGC-1alpha Protects RPE Cells of the Aging Retina against Oxidative Stress-Induced Degeneration through the Regulation of Senescence and Mitochondrial Quality Control. The Significance for AMD Pathogenesis. Int J Mol Sci 19, 2317.
Katz, M. L. & Redmond, T. M. (2001) Effect of Rpe65 knockout on accumulation of lipofuscin fluorophores in the retinal pigment epithelium. Invest Ophthalmol Vis Sci 42, 3023-3030.
Kennedy, C. J. et al. (1995) Lipofuscin of the retinal pigment epithelium: a review. Eye 9, 763-771.
Kim, S. R. et al. (2004) Rpe65 Leu450Met variant is associated with reduced levels of the retinal pigment epithelium lipofuscin fluorophores A2E and iso-A2E. Proc. Natl. Acad. Sci. USA 101, 11668-11672.
Kiser, P. D. et al. (2017) Rational Tuning of Visual Cycle Modulator Pharmacodynamics. J Pharmacol Exp Ther 362, 131-145.
Klein, R. et al. (1999) The prevalence of age-related maculopathy by geographic region and ethnicity. Progress in retinal and eye research 18, 371-389.
Kubota, R. et al. (2012) Safety and effect on rod function of ACU-4429, a novel small-molecule visual cycle modulator. Retina 32, 183-188.
Kubota, R. et al. (2014) Phase 1, dose-ranging study of emixustat hydrochloride (ACU-4429), a novel visual cycle modulator, in healthy volunteers. Retina 34, 603-609.
Liang, F. Q. & Godley, B. F. (2003) Oxidative stress-induced mitochondrial DNA damage in human retinal pigment epithelial cells: a possible mechanism for RPE aging and age-related macular degeneration. Exp Eye Res 76, 397-403.
Lorenz, B. et al. (2004) Lack of fundus autofluorescence to 488 nanometers from childhood on in patients with early-onset severe retinal dystrophy associated with mutations in RPE65. Ophthalmology 2004, 1585-1594.
Lücking, U. (2019) Neglected sulfur(VI) pharmacophores in drug discovery: exploration of novel chemical space by the interplay of drug design and method development. Org Chem Front 6, 1319-1324.
Ma, H. et al. (2020) Inhibition of thyroid hormone signaling protects retinal pigment epithelium and photoreceptors from cell death in a mouse model of age-related macular degeneration. Cell Death Dis 11, 24.
Mäder, P. & Kattner, L (2020) Sulfoximines as Rising Stars in Modern Drug Discovery? Current Status and Perspective on an Emerging Functional Group in Medicinal Chemistry. J. Med. Chem. 63, 14243-14275.
Maeda, A. et al. (2006) Effects of potent inhibitors of the retinoid cycle on visual function and photoreceptor protection from light damage in mice. Mol Pharmacol 70, 1220-1229.
Maeda, A. et al. (2008) Retinopathy in mice induced by disrupted all-trans-retinal clearance. J Biol Chem 283, 26684-26693.
Maeda, A. et al. (2009 A) Limited roles of Rdh8, Rdh12, and Abca4 in all-trans-retinal clearance in mouse retina. Invest Ophthalmol Vis Sci 50, 5435-5443.
Maeda, A. et al. (2009 B) Involvement of all-trans-retinal in acute light-induced retinopathy of mice. J Biol Chem 284, 15173-15183.
Maeda, A. et al. (2012) Primary amines protect against retinal degeneration in mouse models of retinopathies. Nat Chem Biol 8, 170-178.
Maiti, P. et al. (2006) Small molecule RPE65 antagonists limit the visual cycle and prevent lipofuscin formation. Biochemistry 45, 852-860.
Marlhens, F. et al. (1998) Autosomal recessive retinal dystrophy associated with two novel mutations in the RPE65 gene. European Journal of Human Genetics 6, 5.
Mata, N. L. et al. (2013) Pharmacodynamics of visual cycle modulation in the treatment of GA. Retinal Physician 10, 22-25.
Moiseyev, G. et al. (2003) Retinyl esters are the substrate for isomerohydrolase. Biochemistry 42, 2229-2238.
Moiseyev, G. et al. (2005) RPE65 is the isomerohydrolase in the retinoid visual cycle. Proc. Natl. Acad. Sci. USA 102, 12413-12418.
Nikolaeva, O. et al. (2009) Purified RPE65 shows isomerohydrolase activity after reassociation with a phospholipid membrane. FEBS J. 276, 3020-3030.
Palczewski, K. (2010) Retinoids for treatment of retinal diseases. Trends Pharmacol Sci 31, 284-295.
Qiu, F. et al. (2017) Therapeutic Effects of PPARalpha Agonist on Ocular Neovascularization in Models Recapitulating Neovascular Age-Related Macular Degeneration. Invest Ophthalmol Vis Sci 58, 5065-5075.
Qiu, F. et al. (2019) Fenofibrate-Loaded Biodegradable Nanoparticles for the Treatment of Experimental Diabetic Retinopathy and Neovascular Age-Related Macular Degeneration. Mol Pharm 16, 1958-1970.
Radu, R. A. et al. (2003) Treatment with isotretinoin inhibits lipofuscin accumulation in a mouse model of recessive Stargardt's macular degeneration. Proc. Natl. Acad. Sci. USA 100, 4742-4747.
Radu, R. A. et al. (2005) Reductions in serum vitamin A arrest accumulation of toxic retinal fluorophores: a potential therapy for treatment of lipofuscin-based retinal diseases. Investigative ophthalmology & visual science 46, 4393-4401.
Radu, R. A. et al. (2008) Accelerated accumulation of lipofuscin pigments in the RPE of a mouse model for ABCA4-mediated retinal dystrophies following Vitamin A supplementation. Invest Ophthalmol Vis Sci 49, 3821-3829.
Radu, R. A. et al. (2011) Complement system dysregulation and inflammation in the retinal pigment epithelium of a mouse model for Stargardt macular degeneration. J Biol Chem 286, 18593-18601.
Rajala, A. et al. (2018) Pyruvate kinase M2 isoform deletion in cone photoreceptors results in age-related cone degeneration. Cell Death Dis 9, 737.
Rajala, A. et al. (2018) Pyruvate kinase M2 regulates photoreceptor structure, function, and viability. Cell Death Dis 9, 240.
Redmond, T. M. et al. (1998) Rpe65 is necessary for production of 11-cis-vitamin A in the retinal visual cycle. Nat Genet 20, 344-351.
Reid, M. J. et al. (2019) Oxidative deamination of emixustat by human vascular adhesion protein-1/semicarbazide-sensitive amine oxidase. Drug Metab Dispos 47, 504-515.
Roberts, L. J. et al. (2012) Stargardt macular dystrophy: common ABCA4 mutations in South Africa—establishment of a rapid genetic test and relating risk to patients. Mol Vis 18, 280-289.
Rosenfeld, P. J. et al. (2018) Emixustat hydrochloride for geographic atrophy secondary to age-related macular degeneration: a randomized clinical trial. Ophthalmology 125, 1556-1567.
Rozanowska, M. et al. (2005) Light-induced damage to the retina: role of rhodopsin chromophore revisited. Photochem Photobiol 81, 1305-1330.
Sawada, O. et al. (2014) All-trans-retinal induces Bax activation via DNA damage to mediate retinal cell apoptosis. Exp Eye Res 123, 27-36.
Shin, Y. et al. (2018) A novel RPE65 inhibitor CU239 suppresses visual cycle and prevents retinal degeneration. Biochim Biophys Acta Mol Basis Dis 1864, 2420-2429.
Sieving, P. A. et al. (2001) Inhibition of the visual cycle in vivo by 13-cis retinoic acid protects from light damage and provides a mechanism for night blindness in isotretinoin therapy. Proc. Natl. Acad. Sci. USA 98, 1835-1840.
Sparrow, J. R. et al. (2003) A2E, a byproduct of the visual cycle. Vision research 43, 2983-2990.
Sparrow, J. R. & Boulton, M. (2005) RPE lipofuscin and its role in retinal pathobiology. Exp Eye Res 80, 595-606.
Travis, G. H. et al. (2007) Diseases caused by defects in the visual cycle: Retinoids as potential therapeutic agents. Annu Rev Pharmacol 47, 469-512.
Walia, S. & Fishman, G. A. (2009) Natural history of phenotypic changes in Stargardt macular dystrophy. Ophthalmic Genet 30, 63-68.
Wenzel, A. et al. (2001) The Rpe65 Leu450Met variation increases retinal resistance against light-induced degeneration by slowing rhodopsin regeneration. The Journal of neuroscience: the official journal of the Society for Neuroscience 21, 53-58.
Wu, Y. et al. (2019) The cGAS/STING pathway: a sensor of senescence-associated DNA damage and trigger of inflammation in early age-related macular degeneration. Clin Interv Aging 14, 1277-1283.
Yu, G. et al. (2021) Peptide derivatives of retinylamine prevent retinal degeneration with minimal side effects on vision in mice. Bioconjug Chem 32, 572-583.
Zhou, J. et al. (2006) Complement activation by photooxidation products of A2E, a lipofuscin constituent of the retinal pigment epithelium. Proc Natl Acad Sci USA 103, 16182-16187.
Zhou, J. et al. (2009) Complement activation by bisretinoid constituents of RPE lipofuscin. Investigative ophthalmology & visual science, 50, 1392-1399.

What is claimed is:

1. A compound having the structure: 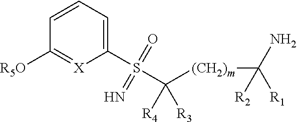
wherein
X is CR₆ or N,
wherein R₆ is H or halogen;
m represents an integer from 0-2;
R₁ is —H, -(alkyl), -(cycloalkyl) or -D;
R₂ is —H, -(alkyl), -(cycloalkyl) or -D or
R₁ and R₂ together form a—(CH₂)ₙ—, wherein n represents an integer from 2 to 5;
R₃ is —H, -(alkyl), -(cycloalkyl) or -D or
R₁ and R₃ together form a —(CH₂)ₒ—, wherein o represents an integer from 1 to 4;
R₄ is —H, -(alkyl), -(cycloalkyl) or -D; and
R₅ is —H, -(alkyl), -(alkylcycloalkyl) or -(alkylheterocycloalkyl),
or a pharmaceutically acceptable salt thereof.

2. The compound of claim 1 having the structure: 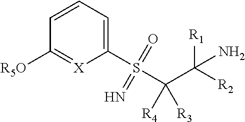
wherein
X is CR₆ or N,
wherein R₆ is H or halogen;
R₁ is —H, -(alkyl), -(cycloalkyl) or -D;
R₂ is —H, -(alkyl), -(cycloalkyl) or -D or
R₁ and R₂ together form a —(CH₂)ₙ—, wherein n represents an integer from 2 to 5;
R₃ is —H, -(alkyl), -(cycloalkyl) or -D or
R₁ and R₃ together form a —(CH₂)ₒ—, wherein o represents an integer from 1 to 4;
R₄ is —H, -(alkyl), -(cycloalkyl) or -D; and
R₅ is —H, -(alkyl), -(alkylcycloalkyl) or -(alkylheterocycloalkyl),
or a pharmaceutically acceptable salt thereof.

3. The compound of claim 1, wherein
X is CR₆ or N,
wherein R₆ is H or halogen;
R₁ is —H, -(alkyl), -(cycloalkyl) or -D;
R₂ is —H, -(alkyl), -(cycloalkyl) or -D;
R₃ is —H, -(alkyl), -(cycloalkyl) or -D;
R₄ is —H, -(alkyl), -(cycloalkyl) or -D; and
R₅ is —H, -(alkyl), -(alkylcycloalkyl) or -(alkylheterocycloalkyl),
or
X is CR₆ or N,
wherein R₆ is H or F;
m represents an integer from 0-2;
R₁ is —H, —CH₃ or -D;
R₂ is —H, —CH₃ or -D or
R₁ and R₂ together form a—(CH₂)ₙ—, wherein n represents an integer from 2 to 5;
R₃ is —H, —CH₃ or -D or
R₁ and R₃ together form a —(CH₂)ₒ—, wherein o represents an integer from 1 to 4;
R₄ is —H, —CH₃ or -D; and
R₅ is —H, 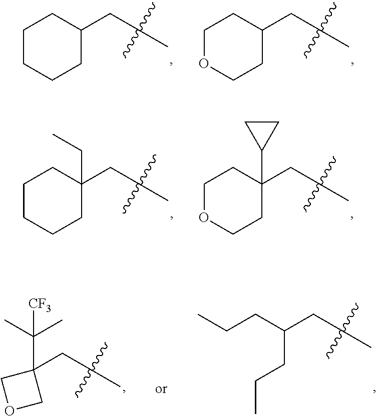
or a pharmaceutically acceptable salt thereof.

4. (canceled)

5. The compound of claim 3, wherein: R₁ is H, —CH₃ or D; and/or R₂ is H, —CH₃ or D;
and/or R₃ is H, —CH₃ or D; and/or R₄ is H, —CH₃ or D, or
wherein: R₁, R₂, R₃ and R₄ are each H; or R₁, R₂, R₃ and R₄ are each D; or R₁ is —CH₃, and R₂, R₃ and R₄ are each H.

6. (canceled)

7. The compound of claim 5, wherein:
X is CH, CF or N; and/or
R₅ is

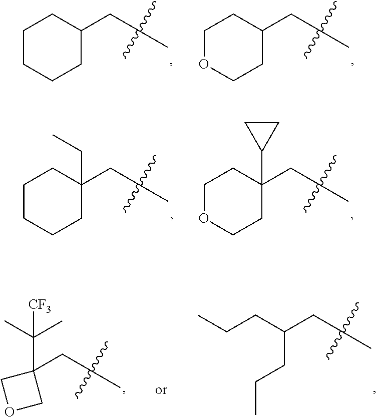

8. The compound of claim 7 having the structure: 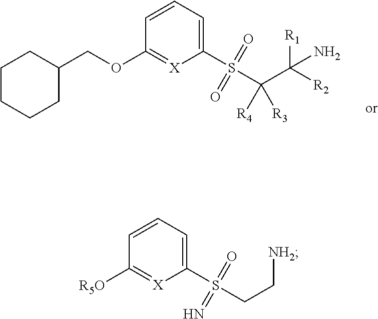
or a pharmaceutically acceptable salt thereof.

9. The compound of claim 2, wherein
R₁ and R₂ together form a—(CH₂)ₙ—, wherein n represents an integer that is: 2; or 3; or 4; or 5.

10. The compound of claim 9 having the structure: 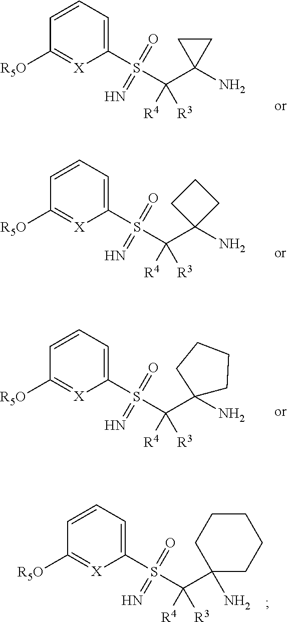
or a pharmaceutically acceptable salt thereof.

11. The compound of claim 1, wherein
R₁ and R₃ together form a —(CH₂)ₒ—, wherein o represents an integer that is: 1; or 2; or 3; or 4.

12. The compound of claim 11, wherein:
m is 1; and/or
the compound has the structure: 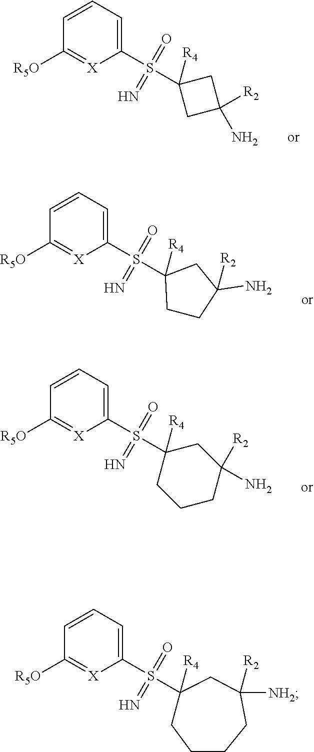
or a pharmaceutically acceptable salt thereof.

13. (canceled)

14. A compound having the structure: 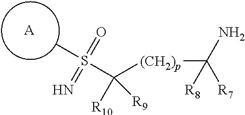
wherein
p represents an integer from 0-2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
A is 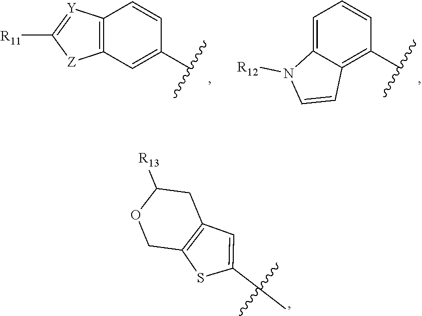
wherein
Y is CH or N,
Z is NR₁₄, O or S,
wherein R₁₄ is H, -(alkyl), -(cycloalkyl), -(alkylcycloalkyl) or -(alkylheterocycloalkyl);
R₁₁ is H, -(alkyl), -(cycloalkyl), -(alkylcycloalkyl) or -(alkylheterocycloalkyl),
R₁₂ is H, -(alkyl), -(cycloalkyl), -(alkylcycloalkyl) or -(alkylheterocycloalkyl),
R₁₃ is H, -(alkyl), -(cycloalkyl), -(alkylcycloalkyl) or -(alkylheterocycloalkyl),
or a pharmaceutically acceptable salt thereof.

15. The compound of claim 14 having the structure: 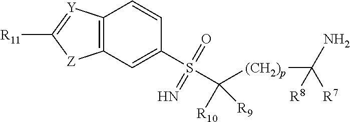
wherein
Y is CH or N;
Z is NR₁₄, O or S,
wherein R₁₄ is H, -(alkyl), -(cycloalkyl), -(alkylcycloalkyl) or -(alkylheterocycloalkyl);
p represents an integer from 0-2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₁ is H, -(alkyl), -(cycloalkyl), -(alkylcycloalkyl) or -(alkylheterocycloalkyl), 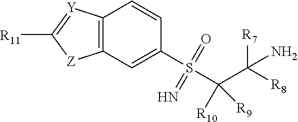
wherein
Y is CH or N;
Z is NR₁₄, O or S,
wherein R₁₄ is H, -(alkyl), -(alkylcycloalkyl) or -(alkylheterocycloalkyl);
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₁ is H, -(alkyl), -(alkylcycloalkyl) or -(alkylheterocycloalkyl),
or a pharmaceutically acceptable salt thereof.

16. (canceled)

17. The compound of claim 11,
wherein
Y is CH or N;
Z is NR₁₄, O or S,
wherein R₁₄ is H, -(alkyl), -(alkylcycloalkyl) or -(alkylheterocycloalkyl);
R₇ is —H, -(alkyl), -(cycloalkyl) or -D;
R₈ is —H, -(alkyl), -(cycloalkyl) or -D;
R₉ is —H, -(alkyl), -(cycloalkyl) or -D;
R₁₀ is —H, -(alkyl), -(cycloalkyl) or -D; and
R₁₁ is H, -(alkyl), -(alkylcycloalkyl) or -(alkylheterocycloalkyl),
or
Y is CH and Z is NR₁₄; or Y is N and Z is O; or Y is CH and Z is O; or Y is CH and Z is S,
or a pharmaceutically acceptable salt thereof.

18. (canceled)

19. The compound of claim 14 having the structure: 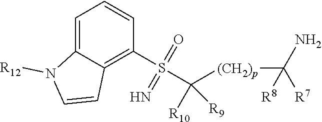
wherein
p represents an integer that is 0, 1 or 2;
R₇ is —H, -(alkyl), -(cycloalkyl) or -D;
R₈ is —H, -(alkyl), -(cycloalkyl) or -D or
R₇ and R₈ together form a —(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₂ is H, -(alkyl), -(cycloalkyl), -(alkylcycloalkyl) or -(alkylheterocycloalkyl), or 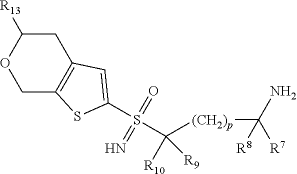
wherein
p represents an integer that is 0, 1 or 2;
R₇ is —H, -(alkyl), -(cycloalkyl) or -D;
R₈ is —H, -(alkyl), -(cycloalkyl) or -D or
R₇ and R₈ together form a—(CH₂)_{q}—, wherein q represents an integer from 2 to 5;
R₉ is —H, -(alkyl), -(cycloalkyl) or -D or
R₇ and R₉ together form a —(CH₂)ᵣ—, wherein r represents an integer from 1 to 4;
R₁₀ is —H, -(alkyl), -(cycloalkyl) or -D; and
R₁₃ is H, -(alkyl), -(cycloalkyl), -(alkylcycloalkyl) or -(alkylheterocycloalkyl),
or a pharmaceutically acceptable salt thereof.

20. (canceled)

21. The compound of claim 14, wherein: R₇ is H,
—CH₃ or D; and/or R₈ is H, —CH₃ or D; and/or R₉ is H, —CH₃ or D; and/or R₁₀ is H, —CH₃ or D, or
R₇, R₈, R₉ and R₁₀ are each H; or R₇, R₈, R₉ and R₁₀ are each D; or R₇ is —CH₃, and R₈, R₉ and R₁₀ are each H, or
R₇ and R₈ together form a —(CH₂)_{q}—, wherein q represents an integer that is: 2; or 3; or 4; or 5, or
R₇ and R₉ together form a —(CH₂)ᵣ—, wherein r represents an integer that is: 1; or 2; or 3; or 4;
wherein p is 1.

22-25. (canceled)

26. The compound of claim 14,
wherein R₁₁ is H, —CH₃, 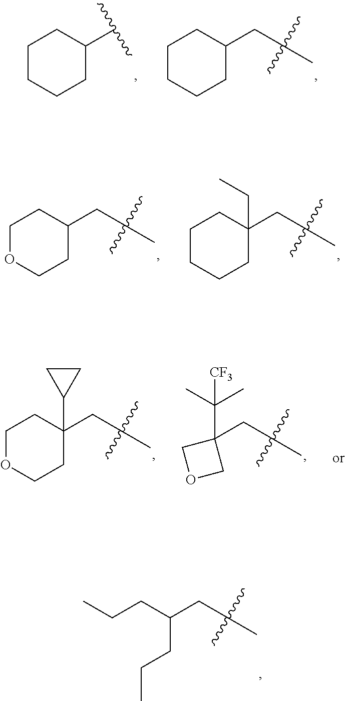
or
R₁₂ is H, —CH₃, 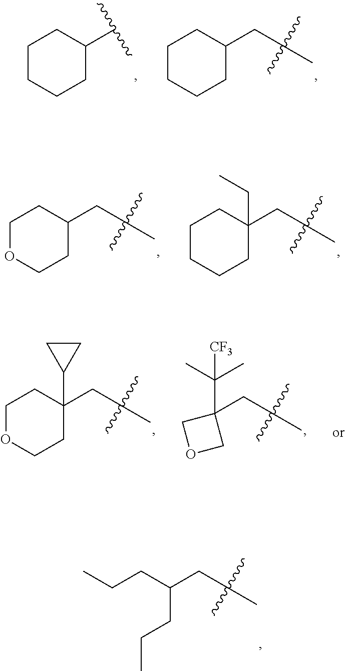
or
R₁₃ is H, —CH₃, 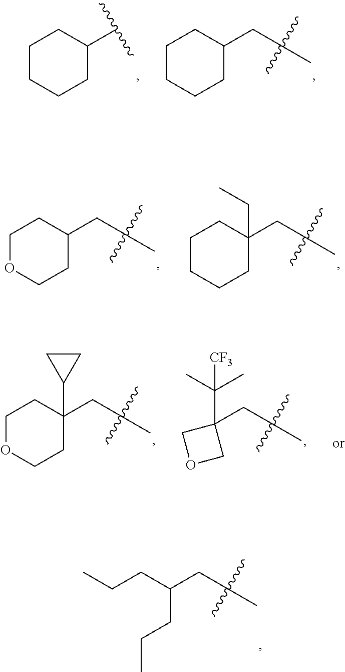
or
R₁₄ is H, —CH₃,

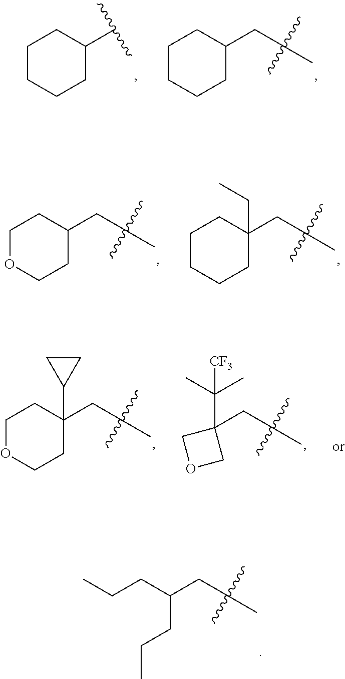

27-29. (canceled)

30. A compound having the structure: 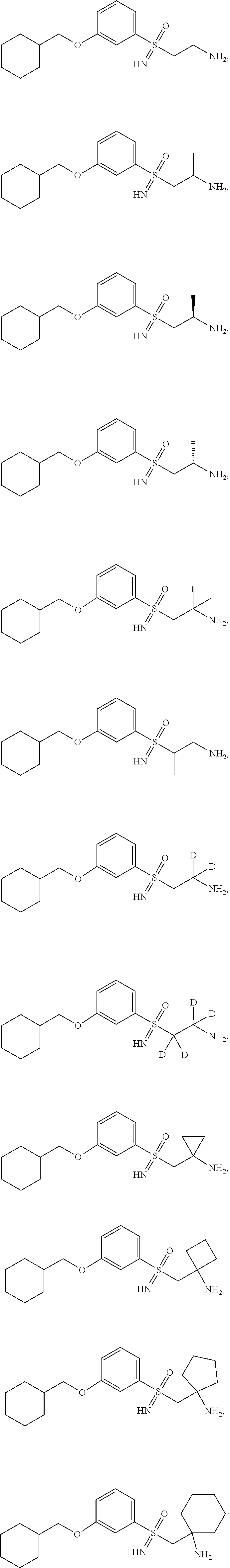 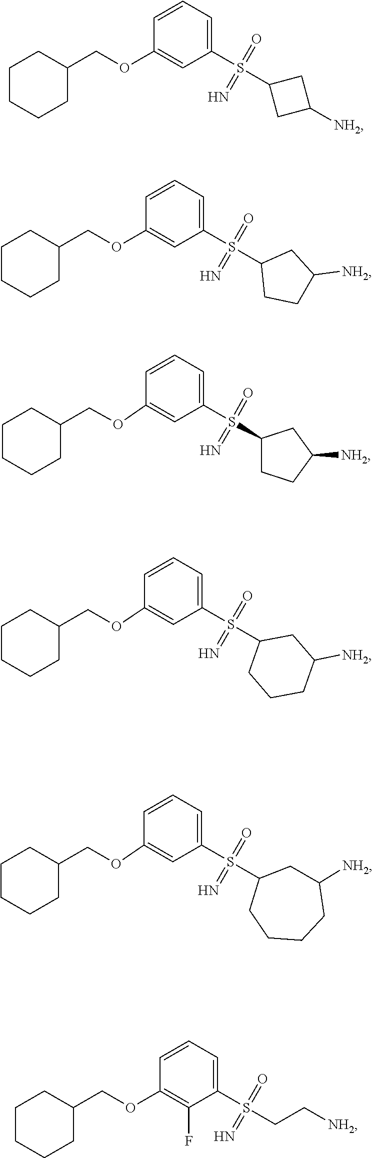 [SMILES: N=S(=O)(c1cccc(OCC2CCCCC2)c1)C1CC(N)C1 ; N=S(=O)(c1cccc(OCC2CCCCC2)c1)C1CCC(N)C1 ; N=S(=O)(c1cccc(OCC2CCCCC2)c1)[C@@H]1CC[C@H](N)C1 ; N=S(=O)(c1cccc(OCC2CCCCC2)c1)C1CCCC(N)C1 ; N=S(=O)(c1cccc(OCC2CCCCC2)c1)C1CCCCC(N)C1 ; N=S(=O)(CCN)c1cccc(OCC2CCCCC2)c1F] 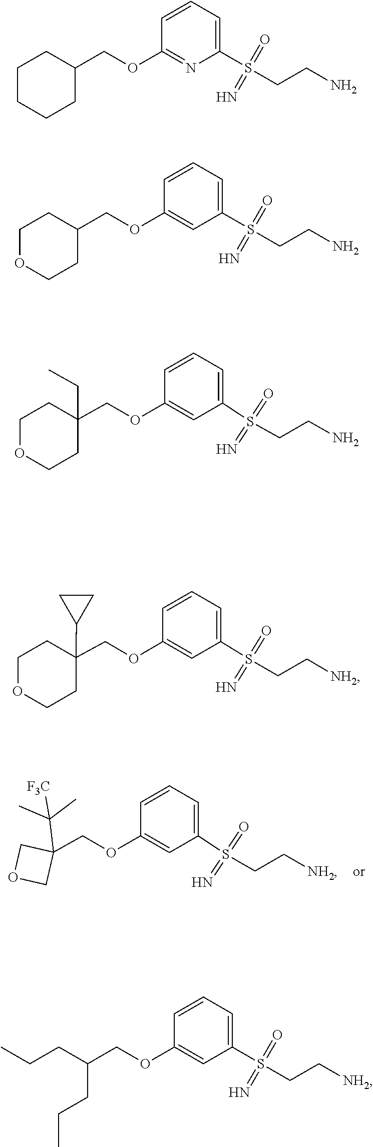 [SMILES: N=S(=O)(CCN)c1cccc(OCC2CCCCC2)n1 ; N=S(=O)(CCN)c1cccc(OCC2CCOCC2)c1 ; CCC1(COc2cccc(S(=N)(=O)CCN)c2)CCOCC1 ; N=S(=O)(CCN)c1cccc(OCC2(C3CC3)CCOCC2)c1 ; CC(C)(C(F)(F)F)C1(COc2cccc(S(=N)(=O)CCN)c2)COC1 ; CCCC(CCC)COc1cccc(S(=N)(=O)CCN)c1] 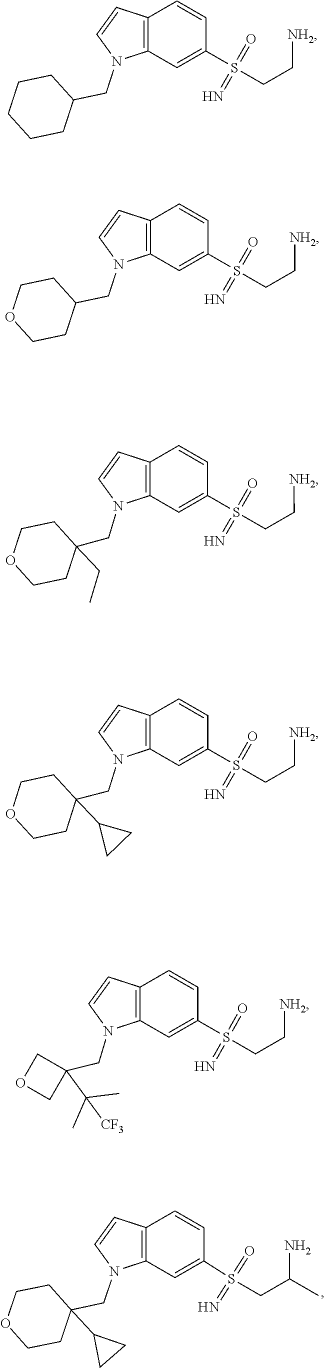 [SMILES: N=S(=O)(CCN)c1ccc2ccn(CC3CCCCC3)c2c1 ; N=S(=O)(CCN)c1ccc2ccn(CC3CCOCC3)c2c1 ; CCC1(Cn2ccc3ccc(S(=N)(=O)CCN)cc32)CCOCC1 ; N=S(=O)(CCN)c1ccc2ccn(CC3(C4CC4)CCOCC3)c2c1 ; CC(C)(C(F)(F)F)C1(Cn2ccc3ccc(S(=N)(=O)CCN)cc32)COC1 ; CC(N)CS(=N)(=O)c1ccc2ccn(CC3(C4CC4)CCOCC3)c2c1] 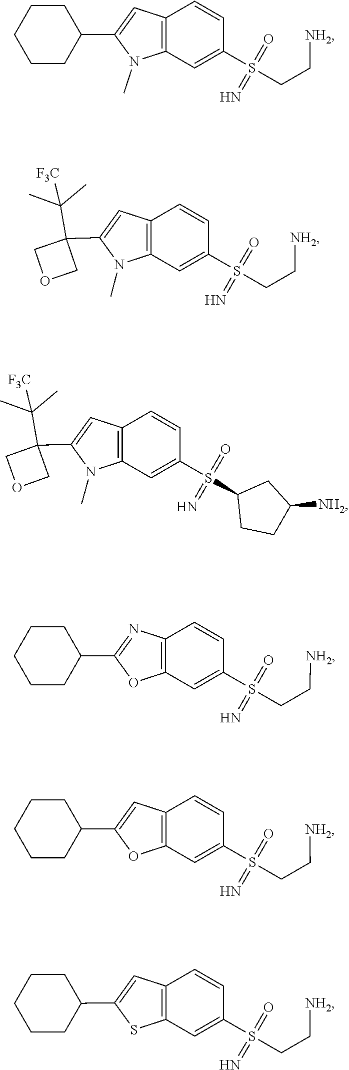 [SMILES: Cn1c(C2CCCCC2)cc2ccc(S(=N)(=O)CCN)cc21 ; Cn1c(C2(C(C)(C)C(F)(F)F)COC2)cc2ccc(S(=N)(=O)CCN)cc21 ; Cn1c(C2(C(C)(C)C(F)(F)F)COC2)cc2ccc(S(=N)(=O)[C@@H]3CC[C@H](N)C3)cc21 ; N=S(=O)(CCN)c1ccc2nc(C3CCCCC3)oc2c1 ; N=S(=O)(CCN)c1ccc2cc(C3CCCCC3)oc2c1 ; N=S(=O)(CCN)c1ccc2cc(C3CCCCC3)sc2c1] 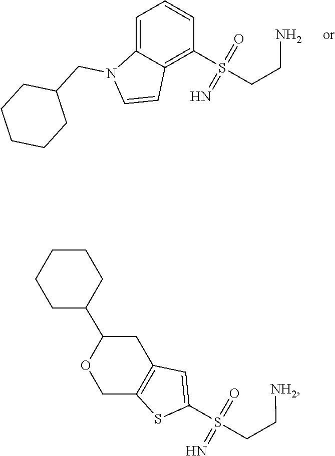 [SMILES: N=S(=O)(CCN)c1cccc2c1ccn2CC1CCCCC1 ; N=S(=O)(CCN)c1cc2c(s1)COC(C1CCCCC1)C2]
or a pharmaceutically acceptable salt thereof.

31. A pharmaceutical composition comprising the compound of claim 30 and a pharmaceutically acceptable carrier.

32. A method of inhibiting RPE65 isomerohydrolase comprising contacting the RPE65 isomerohydrolase with the composition of claim 31.

33. A method for treating a disease characterized by excessive lipofuscin accumulation in the retina in a subject afflicted therewith comprising administering to the subject an effective amount of a compound of the composition of claim 31,
wherein the disease may be further characterized by bisretinoid-mediated macular degeneration;
wherein the amount of the compound is effective to inhibit RPE65 isomerohydrolase in the subject; or the amount of the compound is effective to inhibit the conversion of all-trans retinyl ester to 11-cis retinol in the retinal pigment epithelium in the subject,
wherein the amount of the compound is effective to lower the retinal concentration of a bisretinoid in lipofuscin in the subject,
wherein the bisretinoid is A2E; or the bisretinoid is isoA2E; or the bisretinoid is A2-DHP-PE; or the bisretinoid is atRAL di-PE; or bisretinoid synthesis is reduced through retinaldehyde trapping;
wherein the disease characterized by excessive lipofuscin accumulation in the retina is Age-Related Macular Degeneration, dry (atrophic) Age-Related Macular Degeneration, Stargardt Disease, Best disease, adult vitelliform maculopathy, or Stargardt-like macular dystrophy;
wherein the subject is a mammal,
wherein the mammal is a human,
wherein the compound or pharmaceutical composition is administered to the mammal intravenously, intravitreally, orally or as an oral dosage in the form of a capsule, or tablet.

34-40. (canceled)
